SEQUENCE LISTING (5 sequences)

SEQ ID NO: 1 - DNA, 960 bp, Sarcocystis neurona
  ref <301>: AUTHORS: Ellison,S.P., Omara-Opyene,A.L., Yowell,C.A., Marsh,A.E. and Dame,J.B.
  ref <302>: TITLE: Molecular characterisation of a major 29 kDa surface antigen of Sarcocystis neurona
  ref <303>: JOURNAL: Int. J. Parasitol.
  ref <304>: VOLUME: 32
  ref <305>: ISSUE: 2
  ref <306>: PAGES: 217-225
  ref <307>: DATE: 2002
  ref <308>: DATABASE ACCESSION NUMBER: GenBank/AF401682
  ref <309>: DATABASE ENTRY DATE: 2002-02-21
  ref <313>: RELEVANT RESIDUES: (1)..(960)
  ref <308>: DATABASE ACCESSION NUMBER: GenBank/AF401682
  ref <309>: DATABASE ENTRY DATE: 2002-02-21
  ref <313>: RELEVANT RESIDUES: (1)..(960)
aatgacgagggcggtgctgctgacgtttctgacactctgctccgccagagtgtcccttgtgagggccggagcgccgcctcaagcaacgtgcgccaatggcgaaacgactgttactaagctcggcagctctggcgcactacgaatccactgcccaaataattttcgactcgcgccccgggctgggaatgacgccggtcagatgcaggtctatgcaactgcggttgctgagaatcctgtaaacatacgagacgtcctgcccggcgcatcttacctctctgtacagaacgtcccgaccctcaccgtcccgcaattgcccgccaaagctacgagcgtcttttttcactgccagcagcaacccgacaaccaatgcttcatccaggtagaagtagcgccggctccgcgcctaggtaggtttatatgcattaggagtaggttcatatgcattaggatgcgtactcgtggctcgaatgttccactgcagcgcggtgagattggcggctaaccgggtaaatgtgcgtctttttttgttacgcaggtccgaatacctgcgcggcgctgcagtccacgatcgccttcgaagttcaacaagcgaatgaaacagcagtcttcagctgcggcgagggacttgctgtgttcccgcaaggtagcaaagcgttggatgaagcctgctccaaagagcaggccctacccagtggcgccgctttagctccaaaggatggtgggctccaccttggttttcctcagcttcctcagcaggctatgaagatttgctatatttgtacgaatggtggtgtgcaggcagaggcggcccaacggtgtgaggttcgcatctccgtcgcagcgaacccagacggaagcgttccaggggctaacggagccgcctctctaggagctgccgcacgcagcgcctctgcgttagggttggctctcgttgcaggcgctttcttgcacttttgctaa

SEQ ID NO: 2 - DNA, 1600 bp, Sarcocystis neurona
  ref <301>: AUTHORS: Hyun,C., Gupta,G.D. and Marsh,A.E.
  ref <302>: TITLE: Sequence comparison of Sarcocystis neurona surface antigen from multiple isolates
  ref <303>: JOURNAL: Vet. Parasitol.
  ref <304>: VOLUME: 112
  ref <305>: ISSUE: 1
  ref <306>: PAGES: 11-20
  ref <307>: DATE: 2003
  ref <308>: DATABASE ACCESSION NUMBER: GenBank/AY170620
  ref <309>: DATABASE ENTRY DATE: 2003-06-22
  ref <313>: RELEVANT RESIDUES: (1)..(1600)
  ref <308>: DATABASE ACCESSION NUMBER: GenBank/AY170620
  ref <309>: DATABASE ENTRY DATE: 2003-06-22
  ref <313>: RELEVANT RESIDUES: (1)..(1600)
cggtaatgctgcatcattagggtcagacacgctgtccatttgtcattctcgccagaatgacgagggcgatgctgctgacgatcctgacactctgctccgccagagtgtcattcgtgaacgccgggggggcccgtcaagcgacatgcgaagagggccaaacgactgctactaaactcgagaaccctggcgtactacaactacaatgcccggctcggtatcaactgaatccggctcccgccgcggacgccaacgaaggtatgcaggtctttacaaccgcggctgtcgggaatgctgtggcgcttcaaggtgtcctccccggcgcaacttacgttcgtgcgccaaatggagctggagctacactgaccatcccgcagctgcccccaaaggctgcgagtgtgtttattcaatgccagcaacaaggacaacagggacagtgcttcatcgaggtacaagtagcggggtctccgcgcctaggtaagttctctatgtatgtatgtgtgttcccttcgttgtagctctaatggagcactgcagctccgtgcgactggcagctaacccgggtatcgtgcacttttttttgttacgtaggccgaatacctgcgcggcggtacagtccaggatcgatttcgagattagagcacagaatgaagcagcagtgttcagctgcggcgcgggacgggctccgtttccgcaagcgttggatgatgcctgctcgaaagagcaatccctacccagcggcgtggctttagctccaaaggatgctggttcgttccagcttggctttccgcagctccctcagaaccctctaaagatatgttatatttgcacggaaggtggccagcgggtagatgcggcggatcaaaggtgtgaagttcatatcgccgttgcaggggccgaagccggaggccctacagggcctaccggaggcgcttctgtgggccctgctgcacgcagcgcctctgcattagtcctggctgtcgttgcagccggtttcttccatttttggtgatccattgccggtggaacatctctggttgctccgccccacagatcctgcttactcccacagctgtcagttgggccaacaaccacttcatttcgacgcgccggcgtaacggcgttcagtaacggaacagcttattttggttccatcacggggacatgcataaagtatttcgaggaacttattcatccggtgcagcgtaacatgtgttcagttacgagtggacgcaagtaatttgacgtggttttccattcggtggcagtgtacaggcatggcctcgaatatttgtttttcagtgtaccgctgttgaatatgtgtacttatttatcttgagcgcctgtttcacgtagggggctgcaaaattttttcttgcgctagtcgatgcgctgtgtacgcaaccagagagtgtagtagcttgcggtgagatgacgcagtctaaaaatggttcaacagttgcatttaacagcctccataggttttaatggcgacgtcgccaacggtctgctttttaatccagacggtgtgtcggtttcatatggaactggatccgcagtggttatacgcaatttggtgtcgaacgatcaaatttccttcaca

SEQ ID NO: 3 - DNA, 17 bp, Sarcocystis neurona
ccattccggacgcgggt

SEQ ID NO: 4 - DNA, 15 bp, Sarcocystis neurona
cgtggtgcatggccg

SEQ ID NO: 5 - DNA, 18 bp, Sarcocystis neurona
gcgcgtgcagcccagaac

(19) United States
(12) Patent Application Publication
(10) Pub. No.: US 2005/0260692 A1
(43) Pub. Date: Nov. 24, 2005

(54) Positive selection in the immuno-dominant surface antigen 1 (SAG1) of sarcocystis neurona, agent of EPM: potential as a diagnostic reagent and as a component of a sub-unit vaccine

(75) Inventors: Linda S. Mansfield, Bath, MI (US); Hany M. Elsheikha, Al Gharbyiah Province (EG)

Correspondence Address: Ian C. McLeod;McLEOD & MOYNE, P.C., 2190 Commons Parkway, Okemos, MI, 48864, US

(73) Assignee: Board of Trustees of Michigan State University, East Lansing, MI (US)

(21) Appl. No.: 11/105,236

(22) Filed: Apr. 13, 2005

Related U.S. Application Data
(60) Provisional application No. 60/561,800, filed on Apr. 13, 2004.

(51) Int. Cl. G01N 33/53; G01N 33/569

(52) U.S. Cl. 435/7.22

(57) ABSTRACT

Vaccines against surface antigen gene 1 (SAG1) of Sarcocystis neurona are described, including polypeptide and DNA vaccines. Methods of diagnosis and diagnostic test kits for subtypes of SAG1 are also described. The diagnostic methods and kits are include antibodies, antigens, DNA probes, and PCR primers directed towards regions of nucleotide and amino acid polymorphism to allow clinicians and diagnosticians to distinguish SAG1 genotypes. The methods and kits allow for a more accurate prognosis of the disease.

### CROSS-REFERENCE TO RELATED APPLICATIONS

This application claims priority to provisional Patent Application Ser. No. 60/561,800 filed Apr. 13, 2004.

### STATEMENT REGARDING FEDERALLY SPONSORED RESEARCH OR DEVELOPMENT

Not Applicable

### BACKGROUND OF THE INVENTION

(1) Field of the Invention

The present invention relates generally to prophylactic vaccines and diagnostic tools for equine protozoal myeloencephalitis, and more particularly to subtypes of the surface antigen gene 1 (SAG1) of Sarcocystis neurona. Specifically, the present invention relates to vaccines, diagnostic test kits and methods for detection of Sarcocystis neurona in infected animals.

(2) Description of the Related Art

Sarcocystis neurona is an obligatory intracellular parasite that is responsible for serious economic losses in the horse industry in the United States of America. This parasite is the major causative agent of a nation-wide devastating and often fatal neurological disease of horses in the USA known as equine protozoal myeloencephalitis (EPM). Treatment of this disease is not effective and chances for recovery are very low. Also, the current available vaccine is not effective. Obviously, more must be done in the field of diagnostics and prevention of this disease.

Horses, as all mammals, react to the presence of foreign antigens by producing antibody molecules from its lymphocyte cells. Antibodies have the property of selectively binding to certain distinctive sites, known as determinants on antigens, thereby rendering the antigens innocuous. The antibodies have a physical affinity for specific determinants or epitopes of antigenic material. A reaction between an antibody and a determinant on an antigen for which the antibody is specific results in an adduct, commonly referred to as an “immunocomplex”. The formation of such complexes makes possible a wide variety of assays for antigenic material. Such assays are known generically as immunoassays.

Immunoassays have replaced other procedures used for in vitro diagnostic methods to detect or quantitate a variety of antigens and/or antibodies in fluids and, particularly, body fluids such as blood serum, urine or spinal fluid with important biologic or pharmacologic properties. The high levels of sensitivity and specificity achieved with immunoassays result from the specific, high-affinity, reversible binding of antibodies and antigens, and from the existence of methods for attachment of sensitive detectable labels (radioactive isotopes, fluorophores, ferritin, free radicals, bacteriophages and enzymes) to antibodies or antigens. Enzymes are most commonly used today.

Immunoassay techniques are based upon the complex binding of the antigenic substance being assayed (analyte) with an antibody or antibodies in which one or the other member of the complex may be labeled, permitting the detection and/or quantitative analysis of the target substance by virtue of the label activity associated with the labeled antigen complex or antibody. Immunoassays are generally classified into two groups: the heterogeneous immunoassay in which a labeled antigen or antibody is separated from the labeled antigen-antibody complex before measurement of label activity in either fraction; and the homogeneous immunoassay in which the activity of labeled antigen is measured in the presence of labeled antigen-antibody complex.

Two such diagnostic assay techniques used to determine the presence or amount of antigen in body fluids are generally known as “competitive” assays and “non-competitive” or “sandwich” assays. Typically, in “competitive” assay techniques, an unlabeled antibody or antigen preparation bound to a solid support or carrier is first reacted with a labeled antigen or antibody reagent solution and then with the body fluid sample wherein the antigen or antibody in the sample competes with the labeled antigen for sites on the supported antibody or antigen. The amount of labeled antigen reagent displaced indicates the quantity of antigen present in the fluid sample to be detected.

In the case of a “sandwich” or “non-competitive” assay, a quantity of unlabeled polyclonal or monoclonal antibody or antigen bound to a solid-support or carrier surface, is reacted with a body fluid sample being evaluated for antigens or antibodies, and then, after suitable incubation time and washing, the sample is further incubated with a solution of labeled anti-antibody. The labeled antibody bound to the solid phase in an antibody-antigen-antibody sandwich or the amount of unbound labeled antibody or antigen in the liquid phase would be determined as a measure of the presence of antigen or antibody in the test sample. Thus the analyte can be an antibody or an antigen which has produced antibodies in the host. The more specific the antibodies used, the more specific the information gained by the procedure.

While immunoassay techniques are very precise and sensitive, other diagnostic assay techniques are becoming more widely used. When DNA sequences to an organism are known, genotyping can be performed upon unknown samples by various methods known in the art. Genetic analysis can be performed by Southern blot and DNA hybridization techniques. Polymerase chain reaction based methods are also capable of distinguishing different genotypes in a diagnostic assay.

In addition to the need for diagnostic tests, there are currently no vaccines to protect equids from Sarcocystis neurona and current treatment regimens are effective in only about fifty percent of the equids (Martenuik et al., Proceedings, Conference of Research Workers on Animal Disease, Chicago, Ill., 1997). The U.S. Department of Agriculture (USDA), Animal and Plant Health Inspection Service (APHIS), National Animal Health Monitoring System (NAHMS) of the Needs Assessment Survey (NAS) has designated Equine Protozoal Myeloencephalitis (EPM) as one of the top two infectious diseases of national importance to the horse industry.

Since there are no vaccines for EPM and EPM is a significant health concern of the equine industry, considerable effort has been directed towards developing therapeutic methods for treating EPM. For example, U.S. Pat. No. 5,935,591 to Rossignol et al. describes using thiazolides as a treatment for EPM; U.S. Pat. No. 5,883,095 to Granstrom et al. describes using triazine-based anti-coccidials as a treatment for EPM; U.S. Pat. No. 5,830,893 to Russel et al. describes using triazinediones as a treatment for EPM; U.S. Pat. No. 5,747,476 to Russel describes using a combination of pyrimethamine and a sulfonamide, preferably sulfadiazine in the absence of known therapeutic amounts of trimethoprim as a treatment for EPM; and U.S. Pat. No. 5,925,622 to Rossignol et al. describes using aryl glucuronide of 2-hydroxy-N-(5-nitro-2-thiazolyl)benzamide as a treatment for EPM.

While the related art teach diagnosis and treatment methods for equine protozoal myeloencephalitis utilizing antimicrobials, there still exists a need for a vaccines and improved diagnostic tools for Sarcocystis neurona.

Objects

Therefore, it is an object of the present invention to provide tests for the diagnosis and differentiation of Sarcocystis neurona subtypes.

It is further an object of the present invention to provide vaccines against Sarcocystis neurona.

These and other objects will become increasingly apparent by reference to the following description.

### SUMMARY OF THE INVENTION

The present invention provides a diagnostic test kit comprising one or more primary antibodies each specific for a subtype of a surface antigen gene 1 (SAG1) gene of Sarcocystis neurona, and one or more secondary antibodies conjugated to a particular label which are capable of binding the primary antibodies. In further embodiments of the diagnostic test kit the label is selected from the group consisting of alkaline phosphatase, horseradish peroxidase, fluorescent compounds, luminescent compounds, colloidal gold, radioisotopes, biotin, colored latex, and magnetic particles. In further still embodiments of the diagnostic test kit the one or more primary antibodies are selected from the group consisting of monoclonal antibodies, polyclonal antibodies, and mixtures thereof. In further still embodiments of the diagnostic test kit the one or more primary antibodies are provided by a hybridoma. In further still embodiments of the diagnostic test kit the one or more antibodies are provided from serum.

The present invention also provides a diagnostic test kit comprising one or more polynucleotides specific for subtypes of the surface antigen gene 1 (SAG1) gene of Sarcocystis neurona; and hybridization solution for the binding of the one or more polynucleotides to a sample nucleic acid. In further embodiments of the diagnostic test kit the one or more polynucleotides are selected from the group consisting of oligonucleotides, cDNAs, riboprobes, and mixtures thereof.

The present invention also provides a PCR based diagnostic test kit comprising one or more oligonucleotides specific for a protein-coding region of the surface antigen gene 1 (SAG1) gene of Sarcocystis neurona, one or more oligonucleotides specific for a protein-noncoding region of the surface antigen gene 1 (SAG1) gene of Sarcocystis neurona, and a thermostable DNA polymerase enzyme.

The present invention also provides a method of diagnosing a sample from a horse with equine protozoal myeloencephalitis comprising determining a nucleotide sequence of the surface antigen gene 1 (SAG1) gene of Sarcocystis neurona in the sample, comparing the nucleotide sequence with nucleotide sequences of known surface antigen gene 1 (SAG1) subtypes, and classifying the sample into a Sarcocystis neurona subtype. In further embodiments of the method the nucleotide sequence is determined by DNA sequencing. In further still embodiments of the method the nucleotide sequence is determined by nucleic acid hybridization. In further still embodiments of the method the nucleotide sequence is determined by amplification by polymerase chain reaction (PCR) to produce a DNA followed by cutting of the DNA with a restriction enzyme which recognizes a sequence of interest.

The present invention also provides a method of diagnosing a sample from a horse with equine protozoal myeloencephalitis comprising determining an amino acid sequence of the surface antigen gene 1 (SAG1) gene of Sarcocystis neurona in the sample, comparing the amino acid sequence with amino acid sequences of known surface antigen gene 1 (SAG1) subtypes, and classifying the sample into a Sarcocystis neurona subtype to diagnose the protozoal myeloencephalitis. In further embodiments of the method the amino acid sequence is determined by protein sequencing. In further still embodiments of the method wherein the amino acid sequence is determined by antibody binding.

The present invention also provides a vaccine for active immunization against Sarcocystis neurona comprising one or more polypeptides encoded by a location of positive selection within the surface antigen gene 1 (SAG1) gene of Sarcocystis neurona.

The present invention also provides a vaccine for active immunization against Sarcocystis neurona comprising one or more polynucleotides having a sequence of a location of positive selection within the surface antigen gene 1 (SAG1) gene of Sarcocystis neurona.

The present invention also provides a method of vaccine design comprising determining a region of positive selection within the surface antigen gene 1 (SAG1) gene of Sarcocystis neurona, and selecting one or more polypeptides encoded by the region of positive selection within the surface antigen gene 1 (SAG1) gene to provide the vaccine.

The present invention also provides a method of vaccine design comprising determining a region of positive selection within the surface antigen gene 1 (SAG1) gene of Sarcocystis neurona, and selecting one or more polynucleotides having a sequence of a region of positive selection within the surface antigen gene 1 (SAG1) gene to provide the vaccine.

The present invention also provides a method for making a prognosis related to Sarcocystis neurona infections in clinically infected horses comprising determining a nucleotide or amino acid sequence of the surface antigen gene 1 (SAG1) gene of Sarcocystis neurona in the sample, comparing the nucleotide sequence with nucleotide sequences of known surface antigen gene 1 (SAG1) subtypes, classifying the sample into a Sarcocystis neurona subtype, and predicting the outcome of the infection according to the classification of the Sarcocystis neurona subtype.

The present invention also provides a vaccine for immunization of a mammal against Sarcocystis neurona comprising a polynucleotide which encodes for a surface antigen gene 1 (SAG1) subtype of Sarcocystis neurona. In further embodiments of the vaccine the polynucleotide is operably linked to a promoter which is functional in the mammal.

The present invention also provides a method for eliciting an immune response in an equid against Sarcocystis neurona comprising providing in a carrier solution a polynucleotide sequence encoding for a surface antigen gene 1 (SAG1) subtype of Sarcocystis neurona, and inoculating the equid with the polynucleotide sequence in the carrier solution to illicit the immune response. In further embodiments of the method the polynucleotide is operably linked to a promoter which is functional in the mammal. In still further embodiments of the method the polynucleotide is provided in a vector DNA capable of infecting the equid.

The present invention also provides a diagnostic test kit comprising one or more antibodies each specific for a subtype of surface antigen gene 1 (SAG1) of Sarcocystis neurona; and a signal generating reagent for detecting each of the antibodies bound to the surface antigen.

The present invention provides a method which comprises diagnosing a Sarcocystis neurona infection in an equid which comprises testing with the test kits provided above to determine the presence of the SAG1 gene.

### BRIEF DESCRIPTION OF THE DRAWINGS

FIG. 1 is a maximum parsimonious phylogenetic tree from analysis of 208 phylogenetically informative characters of the SAG1 gene showing relationships among the Sarcocystis neurona isolates (rooted to Toxoplasma gondii). The first of the two numbers at the nodes represent bootstrap resampling results based on maximum parsimony analysis (MP, % of 1000 replicates). The second number represents the bootstrap support using neighbor-joining algorithm (NJ, % of 1000 replicates). Numbers in parentheses after taxon names refer to GenBank accession no., followed by the host and origin of isolate. Tree statistics are length (L)=290, consistency index (CI)=0.997, retention index (RI)=0.998 excluding uninformative characters.

FIGS. 2A and 2B are plots of the variable nucleotide sites (FIG. 2A) and amino acid sites (FIG. 2B) in pairwise comparisons of the two alleles of the SAG1 gene with percentage of polymorphism shown above the domains. Each breakpoint denotes the location of an alignment gap. The three-domain structure model of the Sarcocystis neurona SAG1 gene is drawn above with the intron region shaded in gray. Each vertical line marks the location of a nucleotide (FIG. 2A) or amino acid (FIG. 2B) difference between the two alleles sequences. The arrow marks the signal peptide area.

FIG. 2C is a nucleotide alignment of the SAG1 sequences of two alleles of S. neurona SAG1, AF401682 (SEQ ID NO: 1) and AY170620 (SEQ ID NO: 2), that shows predicted cleavage site for a signal peptide (underlined) as identified by Hyun et al. Vet. Parasitol. 112, 11-20, (2003). Dots indicate sequence identity and dashes indicate alignment gaps.

FIG. 3 is a plot of the number of substitutions per 100 sites for synonymous (p_{S}) and nonsynonymous (p_{N}) sites between HI and H2 SAG1 alleles in a 30-codon sliding window. The difference (p_{N}-pₛ) is a measure of the level of selective constraint on various parts of the molecule.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The term “antibody” as used herein refers to an immunoglobulin molecule with the capacity to bind with a specific antigen as the result of a specific immune response. Immunoglobulins are serum proteins made up of light and heavy polypeptide chains and divisible into classes, which contain within them antibody activities toward a wide range of antigens.

The term “polyclonal antibody” as used herein refers to a mixed population of antibodies made against a particular pathogen or antigen. In general, the population contains a variety of antibody groups, each group directed towards a particular epitope of the pathogen or antigen. To make polyclonal antibodies, the whole pathogen or an isolated antigen is introduced by inoculation or infection into a host which induces the host to make antibodies against the pathogen or antigen.

The term “monoclonal antibody” as used herein refers to antibodies produced by a single line of hybridoma cells all directed towards one epitope on a particular antigen. The antigen used to make the monoclonal antibody can be provided as an isolated protein of the pathogen or the whole pathogen. A hybridoma is a clonal cell line that consists of hybrid cells formed by the fusion of a myeloma cell and a specific antibody-forming cell. In general, monoclonal antibodies are of mouse origin; however, monoclonal antibody also refers to a clonal population of an antibody made against a particular epitope of an antigen produced by phage display technology or method that is equivalent to phage display or hybrid cells of non-mouse origin.

The term “antigen” as used herein refers to a substance which stimulates production of antibody or sensitized cells during an immune response. An antigen includes the whole pathogen or a particular protein or polypeptide of the pathogen. An antigen consists of multiple epitopes, each epitope of which is capable of causing the production of an antibody against the particular epitope. Thus, as used herein antigen also refers to a particular epitope of the protein or polypeptide.

The term “epitope” as used herein refers to an immunogenic region of an antigen which is recognized by a particular antibody molecule. In general, an antigen will possess one or more epitopes, each capable of binding an antibody that recognizes the particular epitope. An antibody can recognize a contiguous epitope which is an epitope that is a linear sequence of amino acids in the antigen molecule, or a non-contiguous epitope which is an epitope that spans non-contiguous amino acids in the antigen which are brought together as a result of the three-dimensional structure of the antigen.

The term “immunoassay” as used herein refers to an analytical method which uses the ability of an antibody to bind a particular antigen as the means for determining the presence of the antigen. An antibody-capture immunoassay is an assay that provides an antigen which is used to detect antibodies against a particular pathogen in a biological sample of a test subject. In general, the antigen is immobilized on a support and is capable of binding an antibody in a biological sample. An antigen-capture assay is an assay that provides an antibody against a particular pathogen which is used to detect a particular antigen in a biological sample from a test subject. In general, the antibody, which can be a monoclonal or polyclonal antibody, is immobilized on a support. The antigen is provided by the biological sample. In a variation of the antigen-capture assay the antibody is mixed with the antigen in the biological sample and the antigen-antibody complex thus formed is captured by a second antibody against the antigen or antibody or both in the antigen-antibody complex which is immobilized on a support. Alternatively, the formation of the antigen-antibody complex is measured in solution.

The term “immunodiffusion” as used herein refers to a method wherein a biological sample is applied to support and allowed to diffuse throughout the support wherein an antigen in the sample binds to an antibody against the antigen which is immobilized to a particular region of the support.

The term “oligonucleotide” as used herein refers to a nucleic acid molecule comprising two or more deoxyribonucleotides or ribonucleotides. The oligonucleotide may be synthetically derived or cloned.

The term “primer” as used herein refers to an oligonucleotide which is capable of initiating synthesis of DNA under polymerase chain reaction conditions in the presence of four nucleoside triphosphates (DATP, dCTP, dGTP, dTTP) and a thermostable enzyme in an appropriate buffer and at suitable temperatures.

The terms “restriction endonuclease” and “restriction enzyme” as used herein refer to bacterial enzymes which cut double-stranded DNA at or near a specific nucleotide sequences.

The term “restriction fragment length polymorphism” (“RFLP”) as used herein refers to the differences in the length and number of restriction fragments formed when a DNA sample from different individuals is digested with a particular restriction endonuclease.

The term “thermostable enzyme” as used herein refers to an enzyme which is heat resistant and is capable of polymerizing DNA after repeated heating cycles. Examples include polymerases extracted from the thermostable bacteria Thermus flavus, Thermus ruber, Thermus thermophilus, Bacillus stearothermophilus, Thermus aquaticus, Thermus lacteus, Thermus rubens, and Methanothermus fervidus.

The term “surface antigen gene 1” or “SAG1” includes all currently known subtypes of the gene including, but not limited to the sequences identified by accession numbers AF401682 (SEQ ID NO: 1), AF397896, AY245695, AY170900, AF480854, AY170620 (SEQ ID NO: 2), AF480853, and AY245696.

Equine protozoal myeloencephalitis (EPM) is the most commonly diagnosed neurological disease in horses in the United States. It is caused by the parasite known as Sarcocystis neurona. Current methods for diagnosis, treatment, and prevention of this disease are not satisfactory. The need for better diagnostic methods and effective prophylactic vaccine is compelling to mitigate the economic impact and devastating effect of this disease on horses and the horse industry. Recent developments, hereby incorporated herein by reference, include U.S. Pat. Nos. 6,153,394 and 6,489,148 to Mansfield et al. which describe immunoassays for Sarcocystis neurona antibodies in equines. Another development includes U.S. Pat. No. 6,344,337 to Mansfield et al., hereby incorporated herein by reference, which describes immunoassays to detect identifying antigens in horses that are infected with Sarcocystis neurona.

Host cell entry of S. neurona sporozoites is most likely to be dependent upon interaction between molecules of the parasite surface coat and those of the host cell surface as determined by the activity of antibodies that inhibit the parasite invasion in tissue culture systems. We detected positive selection in the major surface antigen gene 1 (SAG1) of Sarcocystis neurona. Positive selection is an important determinant of SAG1 gene evolution and indicates that the parts of the gene that under positive selection have more interactions with the host immune systems. Screening the locations of positively selected sites are of use in identifying targets of the immune response and hence aid in vaccine design. SAG1 gene can be used as a single immunogenic prophylactic vaccine for priming protective immune response against S. neurona infections of horses. Also, the sequence diversity of SAG1 gene is relevant for the development of species and genotype specific diagnostic tests, which the clinicians and diagnosticians urgently need.

Antigenic and genetic variation in isolates of this parasite has recently become the target of extensive research. Variations between parasite isolates and how these variations affect the development of EPM disease in horses is clearly of major importance to clinicians. SAG1 gene is the major surface protein-coding gene and corresponds to the 30 KDa of the outer membrane protein of Sarcocystis neurona. By studying divergence in the major surface protein coding gene sequences (SAG1), it is possible to develop a phylogenetic tree that allows related isolates to be grouped and the differences within and between groups studied. With detailed characterization of Sarcocystis neurona isolates at the genetic level, it is possible to find correlation between the clinical presentation of EPM disease in horses and the isolate genotype. This correlation is useful for serological typing of the isolates. Positive selection, which results from interactions of the parasite with the host immune systems, is an important determinant of SAG1 gene evolution and screening the locations of positively selected sites of this gene is useful in identifying targets of the immune response and hence aid in vaccine design. The data provides valuable and promising information which is the basis for: (1) a rapid, highly sensitive, and relatively non-invasive diagnostic test and definitive answers to the question of how S. neurona isolate type influences disease outcome, (2) an effective prophylactic vaccine capable of priming a strong immune response against S. neurona infection in horses.

Diagnostics: Current diagnostic tests can only identify whether the animal is infected or not. It is critical for diagnosticians to have access to rapid and accurate methods for the diagnosis and differentiation of S. neurona subtypes. The diagnostic assays of the present invention can be used for the identification and sub-typing of S. neurona in clinically infected horses. The assays are a valuable diagnostic tool. The diagnostic assays are able not only to detect the infection but also can identify specifically which genotype of the parasite is responsible for the infection. The latter criterion is a matter of great importance because these diagnostic tests allow clinicians and diagnosticians to make a more accurate prognosis and implement the most appropriate treatment, on the basis of the genotype responsible for causing infection.

Vaccination: The evidence that the SAG1 gene has evolved under positive selection pressure and the SAG1 gene has the potential to induce antibodies which neutralize sporozoite-infectivity in tissue culture systems, indicate that this molecule is a good antigen for inclusion into a subunit vaccine preparation of the present invention. Markets that could use this invention include (1) Diagnostic Laboratories (Institutional and Commercial) and (2) Pharmaceutical, Biotechnology, and Animal Health Products Companies.

First, SAG1 is characterized at the genetic level using sequence analyses, the major surface antigen 1 gene (SAG1) in many parasite isolates from horses and other animals from diverse geographic localities in the USA. Sequences are analyzed using multiple computer programs to screen and identify the area of peptide binding motifs of SAG1 gene that are under selection pressure. Examining those amino acid sites under positive selection is a useful way to identify possible epitope regions (areas where antibodies from the host can bind), as some of the positively selected sites that have been detected correspond to important epitopes or highlight regions where others may reside. Not only does such an molecular evolutionary approach shorten the time, labor, and cost of identification of potential targets for vaccine and diagnostic test development, but it ultimately assists our understanding of the immuno-pathogenesis of EPM disease in horses.

Identified herein are two allelic variants within S. neurona parasite isolates originating from different geographical regions. The parasite isolates from diverse geographic localities in the United States are genetically characterized. Methods for accurate and comprehensive analyses of the data are utilized. Molecular Evolutionary Analyses of the data from sequences from various isolates is used to identify useful targets that are used for development of vaccine or diagnostic test.

PCR: The present invention provides a polymerase chain reaction (PCR) based diagnostic test for the identification and differentiation of S. neurona subtypes. The general methods of PCR are well known to those skilled in the art. The PCR methods can be used to test whether an animal is infected or not, and additionally can be used to differentiate between subtypes to allow for a more specific prognosis and treatment regimen tailored to the genotype responsible for the infection.

A specific nucleic acid sequence is synthesized from a nucleic acid containing that sequence as a template which has been isolated from the animal to be tested. The template nucleic acid sample is reacted in the presence of four nucleoside triphosphates, a set of oligonucleotide primers for each nucleic acid sequence subtype to be amplified, a thermostable DNA polymerase, and an appropriate reaction solution preferably buffered at a pH of 7-9. The nucleotide triphosphates include DATP, dCTP, dGTP and dTTP. The concentration of the nucleoside triphosphates in the reaction is usually about 150-200 μM each in the buffer for amplification. MgCl₂ is included in the buffer in an amount of 1.5-2 mM to increase efficiency and specificity of the reaction. Oligonucleotide primers are prepared using any suitable method known in the art, such as phosphotriester and phosphodiester methods whether performed manually or in an automated fashion as described in U.S. Pat. No. 4,458,066.

The PCR techniques can also be utilized for creating nucleic acid sequences of SAG1 subtypes for insertion into suitable expression vectors. The vectors can then be used to transform an appropriate host organism to produce the gene product of the sequence by standard methods of recombinant DNA technology. Alternatively, the nucleic acid sequence can be cloned into a vector suitable for using as a DNA vaccine of the present invention.

The SAG1 gene subtype can be determined by amplifying the sequence and analyzed by gel electrophoresis or Southern blotting techniques known in the art. A small sample of DNA from an animal to be tested is amplified, cut with a restriction enzyme which recognizes polymorphic sequences, and is analyzed one of these techniques. For example, the two alleles of S. neurona SAG1 AF401682 (SEQ ID NO: 1) and AY170620 (SEQ ID NO: 2) can be distinguished by amplification of the region surrounding the TGTACA at nucleotide 279 of AF401682 (SEQ ID NO: 1) and cutting the DNA with the restriction endonuclease BsrGl. The AY170620 (SEQ ID NO: 2) PCR product will not be cut by the enzyme, while the AF401682 (SEQ ID NO: 1) PCR product will be cut by the enzyme. The samples can then be separated by agarose gel electrophoresis and viewed under ultraviolet light to distinguish the SAG1 subtype. Other restriction sites which distinguish the SAG1 subtypes can be designed by methods known in the art. One alternative method of distinguishing the SAG1 subtype is by means of a nucleic acid hybridization method generally known in the art. Small labelled probes such as oligonucleotides, cDNAs, and riboprobes can be hybridized to regions that have variable nucleotide sites and alignment gaps. For example an oligonucleotide specific for AF401682 (SEQ ID NO: 1) can be synthesized by methods known in the art to a region of variable nucleotides such as nucleotides 275-300 of the AF401682 (SEQ ID NO: 1). A person of ordinary skill in the art can then optimize oligonucleotide design and hybridization conditions which will allow Southern blotting and hybridization to distinguish between the SAG1 subtypes.

Southern Blot: DNA samples to be analyzed are size separated by gel electrophoresis typically using an agarose gel in a Tris-borate or Tris-acetate buffer system. The DNA samples are then transferred to and affixed to a membrane such as nitrocellulose, PVDF, or nylon membranes known in the art. The membrane is often pretreated with a prehybridization solution containing sodium dodecyl sulfate and various salts prior to a labeled nucleotide probe being added. The labeled probe can be an oligonucleotide, riboprobe, cDNA or other probes known in the art which is specific to each sequence variation to be detected. The labeled probe is often radioactive to facilitate detection or may be detectable by other means known in the art. The labeled probe is added to a hybridization solution which is applied to the membrane and the membrane and probe are subjected to hybridization conditions that will allow the probe to bind the sample. Typically, hybridization is carried out at about 45 to 65° C. for 0.5-18 hours. Following hybridization, the sample is washed free of unhybridized probe using any means known in the art such as by washing with varying concentrations of standard saline phosphate EDTA (SSPE) (180 mM NaCl, 10 mM NaHPO.sub.4 and 1 M EDTA, pH 7.4) solutions at 45-75° C. for about 10 minutes to one hour. The label can then be detected by using any detection technique known in the art, including autoradiograms or phosphoimaging devices. Southern blotting techniques are described in more detail in Maniatis, T. et al. MOLECULAR CLONING: A LABORATORY MANUAL, Cold Spring Harbor, New York (1982).

SAG1 vaccines: The DNA vaccines against Sarcocystis neurona of the present invention typically comprises one or more circular plasmid vectors and a polynucleotide sequence incorporated into the vector which encodes for a surface antigen gene 1 (SAG1) subtype of Sarcocystis neurona. The SAG1 can be under the transcriptional control of a promoter region active in the cells of the mammal, such as an equid, to be vaccinated. The coding region of SAG1 is preferably followed by a transcriptional termination and polyadenylation site. Preferably, the vector will include an antibiotic resistance gene or other selectable marker gene and a bacterial origin of replication to allow for the DNA to be produced in large quantities by methods known in the art. The DNA vaccine is provided in an accepted pharmaceutical carrier or in a lipid or liposome carrier similar to those disclosed in U.S. Pat. No. 5,703,055 to Felgner herein incorporated by reference. The DNA vaccine can be provided by a variety of methods such as intramuscular injection, intrajet injection, or biolistic bombardment. Making DNA vaccines and methods for their use are provided in U.S. Pat. Nos. 5,589,466 and 5,580,859, both to Felgner which are herein incorporated by reference. Briefly, SAG1 or portions thereof are amplified by PCR utilizing specific oligonucleotide primers which flank the region of interest. The primers can be designed with restriction endonuclease recognition sites incorporated at the 5′ ends of the primers to allow easy cloning into a plasmid vector. Alternatively, SAG1 or portions thereof are provided by traditional genomic library clones known in the art as described by Maniatis, T. et al. MOLECULAR CLONING: A LABORATORY MANUAL, Cold Spring Harbor, New York (1982). A method for producing pharmaceutical grade plasmid DNA is taught in U.S. Pat. No. 5,561,064 to Marquet et al. While not wishing to be limited to one theory, the SAG1 polypeptides which are synthesized in the cells of the mammal to be vaccinated are processed as intracytoplasmic antigens. The DNA vaccine polypeptides thereby may stimulate CD8 T-lymphocyte and/or B lymphocyte immune responses against the Sarcocystis neurona subtypes.

The route of administration for the vaccines of the present invention can include, but is not limited to, intramuscular, intraperitoneal, intradermal, subcutaneous, intravenous, intraarterial, intraocular, and oral as well as transdermal or by inhalation or suppository. The preferred routes of administration include intranasal, intramuscular, intraperitoneal, intradermal, and subcutaneous injection. The vaccine can be administered by means including, but not limited to, syringes, needle-less injection devices, or microprojectile bombardment gene guns (biolistic bombardment).

The vaccines of the present invention are formulated in pharmaceutically accepted carriers according to the mode of administration to be used. One skilled in the art can readily formulated a vaccine that comprises the polypeptide of DNA of the present invention. In cases where intramuscular injection is preferred, an isotonic formulation is preferred. Generally, additives for isotonicity can include sodium chloride, dextrose, mannitol, sorbitol, and lactose. In particular cases, isotonic solutions such as phosphate buffered saline are preferred. The formulations can further provide stabilizers such as gelatin and albumin. In some embodiments, a vaso-constriction agent is added to the formulation. The pharmaceutical preparations according to the present invention are provided sterile and pyrogen free. However, it is well known by those skilled in the art that the preferred formulations for the pharmaceutically accepted carrier which comprise the vaccines of the present invention are those pharmaceutical carriers approved in the regulations promulgated by the United States Department of Agriculture, or equivalent government agency in a foreign country such as Canada or Mexico, for polypeptide, recombinant vector, antibody, and DNA vaccines intended for veterinary applications. Therefore, the pharmaceutically accepted carriers for commercial production of the vaccines of the present invention are those carriers that are already approved or will at some future date be approved by the appropriate government agency in the United States of America or foreign country.

The vaccines of the present invention are generally intended to be a prophylactic treatment which prevents Sarcocystis neurona from establishing an infection in an equid. However, the vaccines are also intended for the therapeutic treatment of equids already infected with Sarcocystis neurona. For example, antibody vaccines of the present invention are suitable for therapeutic purposes. The immunity that is provided by the present invention can be either active immunity of passive immunity and the intended use of the vaccine can be either prophylactic of therapeutic.

With respect to the above, the vaccine that elicits active immunity in a host can be a polypeptide vaccine or a DNA vaccine which produces the polypeptide in a vaccinated host. Alternatively, the vaccine can be a recombinant microorganism vaccine that expresses the SAG1 antigen or a recombinant virus vector that expresses the SAG1 antigen.

Thus, in one embodiment of the present invention, the active immunity is provided by a vaccine that consists of the SAG1 antigen as a fusion polypeptide wherein the amino and/or carboxyl terminus is fused to another polypeptide, preferably a polypeptide that facilitates isolation of the fusion polypeptide. The fusion polypeptide comprising the vaccine is preferably produced in vitro in an expression system from a DNA that encodes the antigens which is in a microorganism such as bacteria, yeast, or fungi; in eukaryote cells such as a mammalian or an insect cell; or in a virus expression vector such as adenovirus, poxvirus, herpesvirus, Simliki forest virus, baculovirus, bacteriophage, or sendai virus. In. particular, suitable bacterial strains for producing the SAG1 antigen as fusion polypeptides include Escherichia coli, Bacillus subtilis, or any other bacterium that is capable of expressing heterologous polypeptides. Suitable yeast for expressing the SAG1 antigen as fusion polypeptides include Saccharomiyces cerevisiae, Schizosaccharomyces pombe, Candida, or any other yeast capable of expressing heterologous polypeptides. Methods for using the aforementioned and the like to produce recombinant polypeptides for vaccines are well known in the art.

For any of the above, transformed host cells are cultured under conditions which produce the SAG1 antigen as fusion polypeptides. The resulting expressed polypeptides can be isolated from the culture medium or cell extracts using purification methods such as gel filtration, affinity purification, ion exchange chromatography, or centrifugation. Furthermore, the present invention further includes polypeptides that comprise only the epitopes of the SAG1 antigen which are responsible for conferring protective immunity against Sarcocystis neurona.

Polynucleotides encoding the SAG1 antigen can be obtained from a genome preparation of Sarcocystis neurona using a polymerase chain reaction (PCR) method that uses DNA oligonucleotide primers which correspond to the nucleotide sequences encoding epitopes such as the regions of positive selection or other portions of the SAG1 antigen. Alternatively, the entire SAG1 antigen can be selected for amplification. In a preferred embodiment, the polynucleotide is in a plasmid and the polynucleotide is operably linked to a promoter which effects the expression of the SAG1 antigen in a microorganism, preferably E. coli. As used herein, the term “operably linked” means that the polynucleotide of the present invention and a DNA containing an expression control sequence, e.g. transcriptional promoter and termination sequences, are situated in a vector or cell such that expression of the antigen encoded by the polynucleotide is regulated by the expression control sequence. Methods of cloning the DNA are well known in the art. Expression of the SAG1 antigen in a microorganism enables the antigen to be produced using fermentation technologies which are used commercially for producing large quantities of recombinant polypeptides.

To facilitate isolation of the SAG1 antigen produced as above, a fusion polypeptide is made wherein the antigen is linked to another polypeptide which enables isolation by affinity chromatography. Preferably, a fusion polypeptide is made using one of the aforementioned expression systems. Therefore, the DNA encoding the SAG1 antigen is linked to a DNA encoding a second polypeptide to produce a fusion polypeptide wherein the amino and/or carboxyl terminus of the antigen is fused to a polypeptide which allows for the simplified recovery of the antigen as a fusion polypeptide. The fusion polypeptide can also prevent the antigen from being degraded during purification. While a vaccine comprising the fusion polypeptide is efficacious, in some instances it can be desirable to remove the second polypeptide after the purification. Therefore, it is also contemplated that the fusion polypeptide comprise a cleavage site at the junction between the antigen and the polypeptide. The cleavage site consists of an amino acid sequence that is cleaved with an enzyme specific for the amino acid sequence of that site. Examples of such cleavage sites that are contemplated include the enterokinase cleavage site which is cleaved by enterokinase, the factor Xa cleavage site which is cleaved by factor Xa, and the GENENASE cleavage site which is cleaved by GENENASE enzyme (New England Biolabs, Beverly, Mass.).

An example of a prokaryote expression system for producing the SAG1 antigen is the glutathione S-transferase (GST) gene fusion system available from Amersham Pharmacia Biotech, Piscataway, N.J., which uses the pGEX-4T-1 expression vector plasmid. The DNA encoding the antigen is fused in frame with the GST gene. The GST part of the fusion polypeptide allows for the rapid purification of the fusion polypeptide using glutathione Sepharose 4B affinity chromatography. After purification, the GST portion of the fusion polypeptide can be removed by cleavage with a site-specific protease such as thrombin or factor Xa to produce a polypeptide free of the GST gene. The antigen free of GST is produced by a second round of glutathione Sepharose 4B affinity chromatography.

Another example for producing the SAG1 antigen is a method which links in-frame with the gene encoding the antigen, codons what encode polyhistidine. The polyhistidine preferably comprises six histidine residues which allows purification of the fusion polypeptide by metal affinity chromatography, preferably nickel affinity chromatography. To produce the native antigen free of the polyhistidine, a cleavage site such as an enterokinase cleavage site is fused in frame between the codons encoding the polyhistidine and the codons encoding the antigen. The native polypeptide free of the polyhistidine is made by removing the polyhistidine by cleavage with enterokinase. The antigen free of the polyhistidine is produced by a second round of metal affinity chromatography. This method was shown to be useful for preparing the LcrV antigen of Y. pestis which was disclosed in Motin et al. Infect. Immun. 64: 4313-4318 (1996), which is hereby incorporated herein by reference. The Xpress System available from Invitrogen, Carlsbad, Calif. is an example of a commercial kit which is available for making and then isolating polyhistidine-polypeptide fusion proteins.

A method further still for producing the SAG1 antigen is disclosed by Motin et al., Infect, Immun. 64: 3021-3029 (1995). Motin et al. disclosed a DNA encoding a fusion polypeptide consisting of the DNA encoding an antigen linked to DNA encoding a portion of protein A wherein DNA encoding an enterokinase cleavage site is interposed between the DNA encoding protein A and the antigen. The protein A enables the fusion polypeptide to be isolated by IgG affinity chromatography, and the antigen free of the protein A is produced by cleavage with an enterokinase. The protein A is then removed by a second round of IgG affinity chromatography.

Another method for producing polypeptide vaccines against Sarcocystis neurona is based on methods disclosed in U.S. Pat. No. 5, 725,863 to Daniels et al., which is hereby incorporated herein by reference. The Daniels method can be used to make the SAG1 vaccine of the present invention which consists of an enterotoxin which has been inserted therein upwards of 100 amino acid residues of the SAG1 antigen. Another method that can be used to make the polypeptide vaccines of the present invention is disclosed in U.S. Pat. No. 5,585,100 to Mond et al., which is hereby incorporated herein by reference, which provides methods for making various fusion polypeptide vaccines. Further methods are disclosed in U.S. Pat. No. 5,589,384 to Liscombe, which is hereby incorporated herein by reference. Finally, the PMAL Fusion and Purification System available from New England Biolabs is another example of a method for making a fusion polypeptide wherein a maltose binding protein is fused to the antigen. The maltose binding protein facilitates isolation of the fusion polypeptide by amylose affinity chromatography. The maltose binding protein can subsequently be released by cleavage with any of the aforementioned cleavage enzymes.

While bacterial methods are used to produce the SAG1 antigen, it can be desirable to produce the antigen in a eukaryote expression system. A particularly useful system is the baculovirus expression system which is disclosed in U.S. Pat. No. 5,229,293 to Matsuura et al., which is hereby incorporated by reference. Baculovirus expression vectors suitable to produce the antigen are the pPbac and pMbac vectors from Stratagene, and the Bac-N-Blue vector, the pBlueBac4.5 vector, pBlueBacHis2-A,B,C and the pMelBac available from Invitrogen, Carlsbad, Calif.

Another eukaryote system useful for expressing the SAG1 antigen is a yeast expression system such as the ESP Yeast Protein Expression and Purification System available from Stratagene. Another yeast expression system is any one of the Pichia based expression systems from Invitrogen. Mammalian expression systems are also embraced by the present invention. Examples of mammalian expression systems are the LacSwitch II system, the pBK phagemid, pXT1 vector system, and the pSG5 vector system from Stratagene; the pTargeT mammalian expresion vector system, the pSI mammalian expression vector, pCI mammalian expression vector, and pAdVantage vectors available from Promega Corporation, Madison, Wisconsin; and the Ecdysone-Inducible Mammalian Expression System, pCDM8, pcDNA1.1, and pcDNA1.1/Amp available from Invitrogen.

Another method for producing the SAG1 antigen in a eukaryote expression system is to insert DNA encoding the antigen into the genome of a eukaryote cell or in a eukaryote virus expression vector such as herpesvirus, poxvirus, or adenovirus to make a recombinant virus that expresses the antigen. The recombinant virus vectors are used to infect mammalian cells wherein the antigens are produced in the cell. U.S. Pat. No. 5,223,424 to Cochran et al., which is hereby incorporated herein by reference, provides methods for inserting genes into herpesvirus expression vectors. U.S. Pat. Nos. 5,338,683 and 5,494,807 to Paoletti et al. and U.S. Pat. No. 5,935,777 to Moyer et al., which are hereby incorporated herein by reference, provide methods for inserting genes into poxvirus expression vectors such as vaccinia virus, entomopoxvirus, and canary poxvirus. In another embodiment, the genes encoding the SAG1 antigen can be inserted into a defective virus such as the herpesvirus amplicon vector which is disclosed in U.S. Pat. No. 5,928,913 to Efstathiou et al., which is hereby incorporated herein by reference. In any of the aforementioned virus vectors, the gene encoding the antigen are operably linked to a eukaryote promoter at the 5′ end of the DNA encoding the protein and a eukaryote termination signal and poly(A) signal at the 3′ end of the gene. Examples of such promoters are the cytomegalovirus immediate-early (CMV) promoter, the Rous sarcoma virus long terminal repeat (RSV-LTR) promoter, the simian virus 40 (SV40) immediate-early promoter, and inducible promoters such as the metallothionein promoter. An example of a DNA having a termination and poly(A) signal is the SV40 late poly(A) region. Another example of a viral expression system suitable for producing the SAG1 antigen is the Sindbis Expression system available from Invitrogen. The used of these commercially available expression vectors and systems are well known in the art.

While subunit vaccines comprising the SAG1 antigen generally provide good humoral protection, it can be desirable to provide the antigen as a component of a live recombinant vector vaccine. Therefore, the present invention further embraces recombinant virus vector vaccines wherein DNA encoding the antigen is inserted into a recombinant virus vector. In one embodiment of the recombinant virus vector vaccine, the DNA encoding the antigen is inserted into a herpesvirus vector according to the method taught by Cochran et al. in U.S. Pat. No. 5,233,424 which is hereby incorporated herein by reference. It is particularly desirable to have a recombinant virus vector vaccine against Sarcocystis neurona that is fetal safe, which allows the vaccine to be given to pregnant mares without affecting the fetus. U.S. Pat. Nos. 5,741,696 and 5,731,188 to Cochran et al., which are hereby incorporated herein by reference, teach methods for making and using live recombinant herpesvirus vaccine vectors which are fetal safe.

Other recombinant virus vector vaccines embraced by the present invention, include but are not limited to, adenovirus, adeno-associated virus, parvovirus, and various poxvirus vectors to express the SAG1 antigen. For example, U.S. Pat. Nos. 5,338,683 and 5,494,807 to Paoletti et al. teach recombinant virus vaccines consisting of either vaccinia virus or canary poxvirus expressing foreign antigens; and U.S. Pat. No. 5,266,313 to Esposito et al. teaches recombinant raccoon poxvirus vectors expressing foreign antigens. Therefore, the present invention embraces recombinant poxvirus vaccines that express the SAG1 antigen mage according to the methods taught in any one of U.S. Pat. Nos. 5,338,683; 5,494,807; and 5,935,777, which are hereby incorporated herein by reference.

While the above refer to DNA sequences encoding the SAG1 antigen, the present invention also includes RNA sequences for encoding the antigen. The present invention further includes vaccines that comprise the SAG1 antigen as a component of a heat-stable spore delivery system made according to the method taught in U.S. Pat. No. 5,800,821 to Acheson et al., which is hereby incorporated herein by reference. Therefore, the present invention provides a genetically engineered bacterial cell containing DNA encoding the SAG1 antigen. When the recombinant bacterial spore vaccine is orally administered to the equid, the spores germinate in the gastrointestinal tract of the animal and the bacteria expresses the antigen which comes into contact with the animal's immune system and elicits an immune response. The vaccine has the advantage of being heat stable, therefore it can be stored at room temperature for an indefinite period of time.

Another embodiment of the Sarcocystis neurona vaccine is a DNA vaccine that elicits an active immune response in an equid. The DNA vaccine consists of DNA having a DNA sequence substantially similar to the DNA sequence that encodes the SAG1 antigen. The DNA encoding the antigen is operably linked at or near its start codon to a promoter that enables transcription of the SAG1 form the DNA when the DNA is in the cells of the equid or other mammal. Preferably, the DNA is in a plasmid. Promoters for expression of DNAs in DNA vaccines are well known in the art and include among others such promoters as the RSV LTR promoter, the CMV immediate early promoter, and the SV40 T antigen promoter. It is further preferred that the DNA is operably linked at or near the termination codon of the sequence encoding antigen to a DNA fragment comprising a transcription termination signal and poly(A) recognition signal. Preferably, the vaccine is in an accepted pharmaceutical carrier or in a lipid or liposome carrier similar to those disclosed in U.S. Pat. No. 5,703,055 to Felgner, which is hereby incorporated herein by reference. The DNA can be provided to the equid by a variety of methods such as intramuscular injection, intrajet injection, or biolistic bombardment. Making DNA vaccines and methods for their use are provided in U.S. Pat. Nos. 5,589,466 and 5,580,859 both to Felgner, which are hereby incorporated herein by reference. Fianlly, a method for producing pharmaceutical grade plasmid DNA is taught in U.S. Pat. No. 5,561,064 to Marquet et al., which is hereby incorporated herein by reference.

One skilled in the art would appreciate that while the polypeptide produced for the polypeptide vaccine or by the DNA vaccine can be the entire SAG1 antigen, the present invention also includes polypeptide, and DNA vaccines wherein the vaccine consists of a subfragment of the antigen which comprises one or more epitopes of th antigen or a DNA encoding on or more epitopes of the antigen.

In another embodiment of the present invention, the vaccine provides passive immunity to Sarcocystis neurona. A vaccine that elicits passive immunity against Sarcocystis neurona consists of polyclonal antibodies or monoclonal antibodies that are against the SAG1 antigen of Sarcocystis neurona. To make a passive immunity vaccine comprising polyclonal antibodies, the SAG1 antigen or one or more epitopes therefrom are injected into a suitable host for preparing the antibodies, preferably the host is a horse, swine, rabbit, sheep, or goat. Methods for producing polyclonal antibody vaccines from these hosts are well known in the art. For example, the antigen is admixed with an adjuvant such as Freund's complete or the less toxic TiterMax adjuvant available from CytRx Corp., Norcross, Georgia, which then are administered to the host by methods well known in the art.

The passive immunity vaccine can comprise one or more monoclonal antibodies against one or more epitopes of the SAG1 antigen. Methods and hybridomas for producing monoclonal antibodies are well known in the art. While monoclonal antibodies can be made using hybridoma technologies, the monoclonal antibodies against the antigen can also be made according to phage display methods such as that disclosed in U.S. Pat. No. 5,977,322 to Marks et al. which is hereby incorporated herein by reference. Equinized antibodies against the antigen can be made according to methods which have been used for humanizing antibodies such as those disclosed in U.S. Pat. Nos. 5,693,762 and 5,693,761 both to Queen et al. which are hereby incorporated herein by reference. A phage display kit that is useful for making monoclonal antibodies is the Recombinant Phage Antibody System available from Amersham Pharmacia Biotech.

SAG1 polypeptide antigen: It is often difficult to protect patients with a single vaccine when there are multiple strains of the organism in the environment. Studies on HIV, for example, suggest that there are at least five genetically distinguishable groups of the virus. Therefore a single virus strain may not adequately protect people against all of the five strains. A multicomponent vaccine to various subtypes of an organism can be produced in situations where there are multiple subtypes. One embodiment of the present invention utilizes multiple subunit polypeptide vaccines against subtypes of Sarcocystis neurona. Polypeptides can be synthesized by methods known in the art including Fmoc or tBoc chemistries using automated methods. Polypeptide can be synthesized to any region of interest in the surface antigen gene 1 (SAG1) Regions under positive selection pressure may be supplied as antigens to provide a vaccine which will protect the mammal to be vaccinated against each subtype.

The polypeptides as described above can also be used as antigens for immunoassay diagnostic kits. Polypeptides which incorporate amino acid sequences in variable regions of SAG1 alleles can be designed by a person skilled in the art to be reactive with sera from animals infected with one subtype of S. neurona and not others. The polypeptides can be incorporated into ELISA diagnostic kits of the present invention to allow for accurate diagnosis and prognosis.

The polypeptides as described above can also be used as antigens for developing monoclonal or polyclonal antibodies. Polypeptides which incorporate amino acid sequences in variable regions of SAG1 alleles can be designed by a person skilled in the art to be reactive with sera from animals infected with one subtype of S. neurona and not others. The polypeptides can be used to immunize animals for the production of antiserum against SAG1. Alternatively, the polypeptides can be used to create monoclonal antibodies from hybridomas specific to SAG1 subtypes by techniques known in the art. These specific monoclonal antibodies may be used to distinguish S. neurona subtypes by various immunological methods including ELISA, Western Blot, fluorescence activated cell sorting, immunoprecipitation, RIA, and immunohistochemistry techniques known in the art including immunofluorescence.

The present invention provides a monoclonal antibody or mixture of monoclonal antibodies against one or more epitopes of the SAG1 antigens to detect whether the antigens are present in the biological sample. Monoclonal antibodies are preferred because they enable antigen-based immunoassays to be performed with a very high degree of specificity and sensitivity. Both ELISA-based and immunodiffusion-based assays are within the scope of the present invention.

Immunoassay: The immunoassay of the present invention can be a solid phase immunoassay or derivative thereof. An example of a solid phase immunoassay is an enzyme-linked immunosorbent assay (ELISA) developed by Engvall et al., Immunochem. 8: 871 (1971) and further refined by others such as Ljunggren et al. J. Immunol. Meth. 88: 104 (1987) and Kemeny et al., Immunol. Today7: 67 (1986). ELISA and its variations are well known in the art.

For example, in an ELISA of the present invention, antigens in a biological sample from an equine suspected of being infected with Sarcocystis neurona form a complex with a monoclonal antibody to the antigens wherein the monoclonal antibody is immobilized on a surface prior to forming the monoclonal antibody-antigen complex. The monoclonal antibody or mixture of monoclonal antibodies is specific for one or more epitopes of the SAG1 antigen. Thus, the monoclonal antibody is immobilized on a surface by methods well known in the art, preferably in the wells of a microtiter plate which is commonly used for ELISA assays. Next, the biological sample is added to the wells containing the bound monoclonal antibodies and the antigen in the biological sample is allowed to bind to the monoclonal antibodies. The biological sample can be provided neat or in a limiting dilution series in a physiological solution. Unbound material in the sample is removed from the immobilized antibody-antigen complex by washing. The complex is then reacted with a second monoclonal antibody that complexes with the antigen to form a second complex consisting of the monoclonal antibody-antigen-second monoclonal antibody. Alternatively, the second antibody can be a polyclonal antibody since it is unlikely that after washing there would be any Sarcocystis sp. antigens that could cross-react with Sarcocystis neurona-specific polyclonal antibodies.

The second complex can be detected when the second monoclonal or polyclonal antibody is conjugated to a reporter ligand such as horseradish-peroxidase or alkaline phosphatase. Alternatively, the second monoclonal or polyclonal antibody can be conjugated to reporter ligands such as a fluorescing ligand, biotin, colored latex, colloidal gold magnetic beads, radioisotopes or the like. Detection of the complex is by methods well known in the art for detecting the particular reporter ligand. In some instances, it is desirable that the monoclonal or polyclonal antibody against the identifying antigens of Sarcocystis neurona is not conjugated to a reporter ligand. In that instance, a third antibody is provided which is conjugated to a reporter ligand and is against the type of antibody comprising the monoclonal or polyclonal antibody, e.g., in the case of the monoclonal antibody, the third antibody is against mouse antibodies.

A variation of the ELISA is disclosed in U.S. Pat. No. 5,079,172 to Hari et al. which is hereby incorporated herein by reference. While Hari et al. discloses spheres coated with antigen, one skilled in the art would recognize that the spheres could be coated with monoclonal antibodies against the SAG1 antigens. Other immunoassays that are suitable for performing the present invention are disclosed in U.S. Pat. No. 5,620,845 to Gould et al.; U.S. Pat. No. 4,486,530 to David et al.; U.S. Pat. No. 5,559,041 to Kang et al.; U.S. Pat. No. 5,656,448 to Kang et al.; U.S. Pat. No. 5,728,587 to Kang et al.; U.S. Pat. No. 5,695,928 to Stewart et al.; U.S. Pat. No. 5,169,789 to Bernstein et al.; U.S. Pat. Nos. 5,177,014, 5,219,725, and 5,627,026 to O° Conner et al.; U.S. pat. No. 5,976,896 to Kumar et al.; U.S. Pat. Nos. 4,939,096 and 4,965,187 to Tonelli; U.S. Pat. No. 5,256,372 to Brooks et al.; U.S. Pat. Nos. 5,166,078 and 5,356,785 to McMahon et al.; U.S. Pat. Nos. 5,726,010, 5,726,013, and 5,750,333 to Clark; U.S. Pat. Nos. 5,518,892, 5,753,456, and 5,620,895 to Naqui et al.; U.S. Pat. Nos. 5,700,655 and 5,985,594 to Croteau et al.; and U.S. Pat. No. 4,786,589 to Rounds et al. The aforementioned U.S. patents are hereby incorporated herein by reference. In all the aforementioned, the immunoassay is modified to detect the SAG1 antigens in a biological sample of an equine, preferably the serum or cerebrospinal fluid.

The immunoassay of the present invention can also be provided as a kit. In one embodiment, the kit provides a microtiter plate or equivalent wherein a series of wells are coated with the monoclonal antibody. A second series of wells are coated with a non-reactive antigen such as bovine serum albumen. The second series of wells serves as a negative control. Optionally, a third series of wells coated with the purified SAG1 antigens is provided. The third series of wells serves as a positive control for the detection method included with the kit. To test a biological sample, the sample is cleared of red blood cells, if present, by standard methods available in any medical laboratory. The sample is serially diluted from a range of neat to 1:1,000. An aliquot of each dilution is dispensed into separate wells of each of the first, second, and third series of wells. The plate is incubated at room temperature for time sufficient for the SAG1 antigens, if present in the sample, to form a complex with the monoclonal antibody, usually 30 minutes to 2 hours. Afterwards, the wells are washed free of unbound material and a ligand-conjugated polyclonal or monoclonal antibody is added to each well. The plate is incubated for approximately 30 minutes or more at room temperature and then the unbound antibodies are washed from the wells. The monoclonal antibody-antigen-ligand-conjugated-antibody complex is detected by a detection method suitable to detect the ligand. The ligand can be a color producing ligand such as alkaline phosphatase or horseradish peroxidase, or a fluorescing compound such as FITC. Preferably, the above method is used to test serum or cerebrospinal fluid from the equine.

Since it is important to be able to test samples in the field for Sarcocystis neurona, the present invention further includes rapid immunodiffusion-based methods, their devices, and kits comprising the same. Therefore, the present invention can be provided as a kit comprising any one of the methods described in U.S. Pat. No. 5,620,845 to Gould et al., U.S. Pat. No. 5,559,041 to Kang et al., U.S. Pat. No. 5,656,448 to Kang et al., U.S. Pat. No. 5,728,587 to Kang et al., U.S. Pat. No. 5,695,928 to Stewart et al., U.S. Pat. No. 5,169,789 to Bernstein et al. U.S. Pat. No. 4,486,530 to David et al., and U.S. Pat. No. 4,786,589 to Rounds et al. While the aforementioned disclose particular rapid immunodiffusion methods, the present invention is not to be construed to be limited to the aforementioned. It is within the scope of the present invention to embrace derivations and modifications of the aforementioned. Thus, in one embodiment of the kit, one or more monoclonal antibodies against one or more epitopes of one or both of the Sarcocystis neurona SAG1 antigens is immobilized on a membrane in a device designed for analyzing a biological sample. A biological sample is applied to the membrane which diffuses throughout the membrane. If the sample contains the identifying Sarcocystis neurona antigens, the identifying antigens will form a complex with the monoclonal antibodies on the membrane. Detection of the antibody-antigen complex is by a calorimetric method incorporated into the device, by immersing the device into a solution that causes a calorimetric reaction, or by reacting with a second monoclonal or polyclonal antibody conjugated to a reporter ligand.

While the above methods have been provided, other immunoassays are also within the scope of the present invention. For example, the present invention comprises an immunoassay comprising the SAG1 antigen coupled to a reporter dye such as 6-carboxyfluorescein (FAM) or 6-carboxy-4,7,2′,7′-tetrachlorofluorescein (TET) and an anti-16-and/or-30-kDa monoclonal antibody coupled to a quencher such as 6-carboxy-N,N,N′,N′-tetramethylrhodamine. The quencher is attached to the monoclonal antibody such that when the labeled antigen binds the antibody, the quencher and reporter dye are in close proximity, and the reporter dye is prevented from fluorescing. Therefore, when a sample does not contain the SAG1 antigen, all of the antigen is bound by the monoclonal antibody. Since the quencher and reporter dyes are in close proximity, the quencher prevents the reporter dye from fluorescing. However, when a sample contains the SAG1 antigen, the SAG1 antigen in the serum competes with the labeled SAG1 antigen for the antibody, which results in some labeled SAG1 antigen molecules remaining unbound. Because these unbound labeled SAG1 antigen molecules are no longer in close proximity to the quencher on the antibody, the reporter dye on these labeled SAG1 antigens will fluoresce. The intensity of the fluorescence is directly proportional to the amount of SAG1 antigen in the sample. The advantage of this embodiment or variations of this embodiment which would be appreciated by those skilled in the art is that it can be performed in a small reaction volume and the results of the assay can be known instantaneously. Suitable devices for detecting the fluorescence include ELISA reading devices that detect the appropriate fluorescing wavelength, or spectrophotometers or fluorometers.

Methods described in U.S. Pat. No. 6,344,337 to Mansfield, et al. are particularly useful for providing purified Sarcocystis neurona antigens for producing polyclonal or monoclonal antibodies, or for providing sufficient quantities of the organism from a biological sample from an equine to unequivocally determine whether an equine was infected with Sarcocystis neurona.

Mansfield, et al. teach the steps of mechanically disrupting a biological sample from an opossum in a physiological solution to make a homogenate. Preferably, the physiological solution is phosphate buffered saline (pH 7.4) and mechanical disruption is by a device such as a Dounce homogenizer. The homogenate is washed with the physiological solution and concentrated by low-speed centrifugation. The homogenate is then resuspended in a digestion solution consisting of pepsin-NaCl-HCl and incubated with frequent mixing at 37° C. for about 1.5 hours. Preferably, the pepsin-NaCl—HCl solution contains 0.65% pepsin (w/v), 0.86% NaCl (w/v), and 1% concentrated hydrochloric acid (v/v).

Afterwards, the semi-digest is washed with the physiological solution and resuspended in cell culture medium containing antibiotics until ready for culturing. Preferably, the culture medium is Hank's balanced salt solution containing penicillin (about 100 units per ml), amikacin (about 100 pg per ml), and amphotericin B (about 1.25 μg per ml).

For cell culturing, the semi-digest is concentrated by low-speed centrifugation, resuspended in a 2.6% hypochlorite solution, and stirred for about 1.5 hours at room temperature. Afterwards, the hypochlorite treated sample is concentrated by low-speed centrifugation and washed with the physiological solution. Next, the washed sample is concentrated and suspended in a digestion solution preferably consisting of 10% trypsin in an alkaline chelating solution (ACS) which consists of 100 mM NaCl, 3 mM KCl, 9 mM Na₂HPO₄, 3 mM Na-citrate, 0.5 mM Na₂EDTA, 0.1% glucose, 0.3% HEPES, 100 units per ml penicillin, and 1.25 μg per ml amphotericin B. After about 1.5 hours at 37° C., the sample is washed with the physiological solution, mechanically sheared and then applied to cell cultures of equine dermal cells. Preferably, the cell cultures are confluent and maintained in Dulbecco's modified Eagle's medium containing L-glutamine, 6% heat-inactivated fetal bovine serum, 100 units per ml penicillin, 100 μg per ml amikacin, and 1.25 μg per ml amphotericin B. The cultures are preferably incubated at 37° C. with 5% CO₂ and the culture medium changed every other day for about seven days, and then weekly thereafter. The cultures are monitored for evidence of Sarcocystis neurona infection, which usually appears by about the 14th day of culture. A suitable equine dermal cell line for culturing Sarcocystis neurona is the ATCC No. CCL-57 equine dermal cell line available from the American Type Culture Collection, 10801 University Boulevard, Manassas, Va. 20110.

The above cell line is also suitable for isolating Sarcocystis neurona from equines as well (Murphy et al., The Conference of Research Workers in Animal Diseases, November 10-11, Chicago, Ill. (1997)). For example, neural tissue from spinal cord or brain are removed and placed in Hank's balanced salts containing antibiotics such as penicillin, amikacin, and amphotericin B and kept at room temperature. Preferably, within two hours of collection, portions of the tissue are minced and ground in a Dounce homogenizer with cell culture medium to make a slurry. Next, the slurry is poured onto confluent equine dermal cells such as ATCC No. CCL-57 and incubated preferably at 37° C. with 5% CO₂ for about 24 hours. The medium is replaced, and then replaced every other day for the first week, and then weekly thereafter. The cultures are monitored for evidence of Sarcocystis neurona infection, which usually appears about the 14th day of culture.

The aforementioned culturing methods enable sufficient Sarcocystis neurona to be available for producing the identifying antigens for making polyclonal or monoclonal antibodies for the immunoassay of the present invention, and for producing sufficient Sarcocystis neurona from a biological sample from an equine to enable identification using the immunoassay of the present invention.

An alternative method for concentrating Sarcocystis neurona antigens in serum and cerebrospinal fluid is using magnetic beads with bound Sarcocystis neurona monoclonal or polyclonal antibodies directed against the SAG1 antigens. Using magnetic beads improves the sensitivity of the antigen test of the present invention. To perform the test, magnetic beads bound to Sarcocystis neurona monoclonal or polyclonal antibodies directed against the SAG1 antigens are incubated with serum or cerebrospinal fluid samples from equines in a container for about 12 hours at 4° C. with stirring. Sarcocystis neurona SAG1 antigens are bound by the antibodies forming an antibody-antigen complex. Afterwards, the magnetic beads are collected by placing a magnet around the container which causes the magnetic beads to be held to the sides of the container. This allows the sample fluid to be removed without loss of the magnetic beads with the bound antigens. After the sample fluid is removed, the magnetic beads with the bound antigens are washed with a buffer such as phosphate buffered saline, pH 7.4 (PBS). Next, the antigen is eluted from the antibodies and beads by removing the magnet and disrupting the antibody-antigen complex by mechanical agitation in PBS or by using a chaotropic reagent. The eluted and concentrated antigen can then be used in the antigen test method of the present invention.

Monoclonal antibodies that recognize and bind to particular epitopes of either the SAG1 antigens of Sarcocystis neurona are produced according to methods that are well known in the art. In particular, Sarcocystis neurona merozoites grown in culture were harvested, the antigens extracted, and the antigens separated using two-dimensional gel electrophoresis: isoelectric focusing followed by SDS-polyacrylamide gel-electrophoresis, or other isolation methods which are well known in the art. The isolated identifying antigens are used to make monoclonal antibodies according to procedures well known in the art such as that described in Antibodies, A Laboratory Manual, eds. Harlow and Lane, Cold Spring Harbor Laboratory Press, Cold Spring Harbor, N.Y. (1988).

While monoclonal antibodies can be made using hybridoma technologies well known in the art, the monoclonal antibodies against the identifying antigens can also be made according to phage display methods such as that disclosed in U.S. Pat. No. 5,977,322 to Marks et al. which is hereby incorporated herein by reference. A phage display kit that is useful for making monoclonal antibodies is the Recombinant Phage Antibody System available from Amersham Pharmacia Biotech (Piscataway, N.J.).

Polyclonal antibodies can be made according to methods taught in Antibodies, A Laboratory Manual, eds. Harlow and Lane, Cold Spring Harbor Laboratory Press, Cold Spring Harbor, N.Y. (1988). For example, polyclonal antibodies can be made by inoculating rabbits, mice, goats, donkey, or horses with live or killed Sarcocystis neurona or the identifying SAG1 antigens. The polyclonal antibodies are isolated from serum from the inoculated animal by ammonium sulfate precipitation to remove primarily the globulin. The precipitated antibodies are preferably further purified by anion exchange chromatography to remove most of the other proteins in the serum. Alternatively, the polyclonal antibodies can be isolated from the cerebrospinal fluid of an animal, preferably the horse, infected naturally or intentionally with live Sarcocystis neurona. Preferably, the antibodies from the cerebrospinal fluid are isolated or further purified by anion exchange chromatography. The purified antibodies are preferably stored in phosphate buffered saline at a neutral pH.

To facilitate making monoclonal and polyclonal antibodies against the identifying SAG1 antigens, it is desirable to produce the SAG1 antigens in vitro. In vitro production of the identifying antigens provides a rapid and simple means for obtaining large quantities of the antigens. Therefore, the SAG1 gene from Sarcocystis neurona that encode the SAG1 antigens are also within the scope of the present invention. The SAG1 gene subtypes are prepared by screening a genomic library according to methods well known in the art. Alternatively, SAG1 gene subtypes are prepared by utilizing polymerase chain reaction (PCR) techniques upon genomic DNA prepared from the by methods well known in the art. Shorter sequences of the SAG1 gene can be prepared by any commercially available oligonucleotide synthesizers utilizing chemistry well known in the art.

Isolation of Sarcocystis neurona can be achieved according to the following method which demonstrates the collection of the intermediate host stage of Sarcocystis neurona. Brown headed cowbirds (Molothrus ater) are collected and euthanized and the carcasses chilled until needed. The muscles are dissected and observed for presence of Sarcocystis sarcocysts, which appear like grains of rice on the surface of the muscle. The sarcocysts are collected and extracted as follows. The muscle is sliced into small chunks and random samples are selected. Then 0.5 g of each sample is immersed in liquid nitrogen and pulverized in a mortar and pestle. Next 6 ml of digestion buffer is added which preferably consists of 100 mM NaCl, 10 mM Tris HCl, 25 mM EDTA, 0.5% sodium dodecyl sulfate, and 2 mg of proteinase K. The samples are incubated overnight in a shaker at 50° C. at 200×g. Next, the nucleic acids are removed from the sample by phenol extraction and recovered by precipitation with ethanol. The nucleic acids are tested for identity to Sarcocystis neurona using a Sarcocystis neurona specific PCR test using primers specific for Sarcocystis neurona SSURNA gene (Fenger et al., J. Parasitol. 81: 199-213 (1995)). The primers are the 3870R Sarcocystis neurona reverse primer 5′-CCATTCCGGACGCGGGT-3′ (SEQ ID NO: 3) and the 1055 eukaryote universal forward primer 5′-CGTGGTGCATGGCCG-3′ (SEQ ID NO: 4). These primers produce a 484 bp product when applied to a Sarcocystis neurona template. Another set of primers can be used to verify the presence of SSURNA DNA in each sample. These primers are the 3475R protist SSURNA reverse primer 5′-GCGCGTGCAGCCCAGAAC-3′ (SEQ ID NO: 5) and the universal primer (SEQ ID NO: 4), which yields a 203 bp product. Samples which test positive for Sarcocystis neurona and no other Sarcocystis sp. are fed to pathogen-free opossums. About one month later after sporocysts are observed in the feces of the inoculated opossums, the mucosa of the small intestine is collected and used to inoculate equine dermal tissue culture cells as described previously. This method provides a means for providing samples of all of the stages of Sarcocystis neurona for use in the present invention.

A variety of methods suitable for producing the SAG1 antigens in large quantities sufficient are well known to those skilled in the art and are described in the prior art, e.g., in Current Protocols in Molecular Biology, section 3.8 (vol.1 1988) or Molecular Cloning: A Laboratory Manual, Second Edition, edited by Sambrook et al. Cold Spring Harbor Laboratory Press, Cold Spring Harbor, N.Y. (1989). For example, the DNA for genes encoding the SAG1 antigens are inserted into a plasmid expression vector according to methods well known to the art. The plasmid vector provides a promoter for transcribing the genes into RNA which is then translated into the antigen. The promoter can be a constitutive promoter, a phage promoter, or an inducible promoter such as the lacZ promoter. To produce the antigens, bacteria such as E. coli are transformed with the plasmid vector. Methods for producing large quantities of antigens in bacterial expression systems are well known in the art.

While bacterial methods are used to produce the SAG1 antigens, it can be desirable to produce the antigens in a eukaryote expression system. A particularly useful system for expressing the SAG1 antigens is the baculovirus expression system which is disclosed in U.S. Pat. No. 5,229,293 to Matsuura et al. which is hereby incorporated herein by reference. Baculovirus expression vectors suitable to produce the SAG1 antigens are the pPbac and pMbac vectors from Stratagene; and the Bac-N-Blue vector, the pBlueBac4.5 vector, pBlueBacHis2-A,B,C, and the pMelBac available from Invitrogen, Carlsbad, Calif.

Another eukaryote system useful for expressing the SAG1 antigens is a yeast expression system such as the ESP Yeast Protein Expression and Purification System available from Stratagene. Another yeast expression system is any one of the Pichia-based Expression systems from Invitrogen. Mammalian expression systems are also embraced by the present invention. Examples of mammalian expression systems are the LacSwitch II system, the pBK Phagemid, pXT1 vector system, and the pSG5 vector system from Stratagene; the pTargeT mammalian expression vector system, the pSI mammalian expression vector, pCI mammalian expression vector, and pAdVantage vectors available from Promega Corporation (Madison, Wis.); and the Ecdysone-Inducible Mammalian Expression System, pCDM8, pcDNA1.1, and pcDNA1.1/Amp available from Invitrogen.

Another method for producing the SAG1 antigens in a eukaryote expression system is to insert the DNA encoding the SAG1 antigens into the genome of the eukaryote cell or in a eukaryote virus expression vector such as herpesvirus, poxvirus, or adenovirus to make a recombinant virus that expresses the SAG1 antigens. The recombinant virus vectors are used to infect mammalian cells wherein the SAG1 antigens are produced in the cell. The SAG1 antigens can be purified using methods well known in the art for purifying antigens. U.S. Pat. No. 5,223,424 to Cochran et al. which is hereby incorporated herein by reference provides methods for inserting genes into herpesvirus expression vectors such as equine herpesvirus. U.S. Pat. Nos. 5,338,683 and 5,494,807 to Paoletti et al. and U.S. Pat. No. 5,935,777 to Moyer et al. which are hereby incorporated herein by reference provide methods for inserting genes into poxvirus expression vectors such as vaccinia virus, entomopoxvirus, and canary poxvirus. Alternatively, the genes encoding the SAG1 antigens can be inserted into a defective virus such as the herpesvirus amplicon vector which is disclosed in U.S. Pat. No. 5,928,913 to Efstathiou et al. which is hereby incorporated herein by reference. In any of the aforementioned virus vectors, the genes encoding the SAG1 antigens are operably linked to a eukaryote promoter at the 5′ end of the DNA encoding the antigen and a eukaryote termination signal and poly(A) signal located at the 3′ end of the gene. Examples of such promoters are the cytomegalovirus immediate-early (CMV) promoter, the Rous sarcoma virus long terminal repeat (RSV-LTR) promoter, the simian virus 40 (SV40) immediate-early promoter, and inducible promoters such as the metallothionein promoter. An example of a DNA having a termination and poly(A) signal is the SV40 late poly(A) region. Another example of a viral expression system suitable for producing the SAG1 antigens of the present invention is the Sindbis Expression system available from Invitrogen (Carlsbad, Calif.). The use of these commercially available expression vectors and systems are well known in the art.

The following examples are intended to promote a further understanding of the present invention.

### EXAMPLE 1

The SAG1 gene is conserved among members of Sarcocystidae and may play an important role in parasite pathogenesis. Patterns of nucleotide polymorphism were used to test the hypothesis that natural selection promotes diversity in different parts of the major surface antigen gene 1 (SAG1) of Sarcocystis neurona. Nucleotide and amino acid sequence analysis of the SAG1 gene from multiple S. neurona isolates identified two alleles. Sequences were identical intra-allele and highly divergent inter-alleles. Also, phylogenetic reconstruction showed sequences clustering into two clades. The ratio of silent substitutions to amino acid replacements provided strong evidence that two short segments in the central and 5′ prime domain of SAG1 have been under positive selection in the divergence of the two alleles, suggesting that it may be important for the evasion of host immune responses and can be a suitable target for the development of a vaccine.

Sarcocystis neurona, the major causative agent of equine protozoal myeloencephalitis (EPM) in horses, is widespread throughout the New World (Dubey et al. Vet. Parasitol. 95, 89-131, 2001). EPM is a serious neurological disease that imposes a serious burden to the horse industry in the United States and for which there is no effective vaccine. Seroprevalence rates among horses can reach 60% (Rossano et al. Prev. Vet. Med. 57, 7-13, 2003) in Michigan and 89.2% in Oklahoma (Bentz et al. J. Vet. Diagn. Invest. 6, 597-600, 2003). The design of an effective vaccine is complicated by the fact that field isolates differ in immunogenicity (Mansfield et al. Vet. Parasitol. 95, 167-78, 2001; Marsh et al. Vet. Parasitol. 95, 143-54, 2001; Marsh et al. Parasitol. Res. 88, 501-6, 2002). Additionally, genetic information for the distinction of S. neurona isolates is lacking.

S. neurona has an immunodominant surface antigen gene 1 (SAG1) which appears to occur as a single copy and which encodes a major antigenic surface protein of about 29 kDa (Ellison et al. Int. J. Parasitol. 32, 217-25, 2002). SAG1 has been suggested as a diagnostic marker for phylogenetically related Sarcocystidaen species from different geographical areas (Hyun et al. Vet. Parasitol. 112, 11-20, 2003). The genetically closely related organism Toxoplasma gondii has an immunodominant major surface antigen P30, which has been extensively studied with a view to the development of a vaccine (Bonenfant et al. Infect. Immun. 69, 1605-12, 2001; Letscher-Bru et al. Infect. Immun. 71,6615-19, 2003).

Positive selection and sequence polymorphism are among the major factors that determine how the gene has evolved and consequently may be important for the development of a vaccine. Synonymous (silent) mutations are largely invisible to natural selection, while nonsynonymous (amino acid-altering) mutations can be under strong selective pressure (Akashi, Genetics 139, 1067-76, 1995). The rates for nonsynonymous (p_{N}) and synonymous (p_{S}) mutations were defined as the numbers of substitutions per site. Comparison of the nonsynonymous/synonymous substitution rate ratio (ω=d_{N}/d_{S}) provides a powerful tool for understanding the effect of natural selection on molecular sequence evolution and can be used as an indicator of the selective pressure at the protein level (Ohta, Annual Rev. Ecol. Syst. 23, 263-86, 1993). In general, amino acid sites in a protein are expected to be under different selective pressures and have different underlying ω ratios where ω=1 indicates neutral mutations, ω<1 means purifying selection, and ω>1 indicates positive selection. Also, nonsynonymous mutations offer fitness advantages to the protein and have higher fixation probabilities than synonymous mutations.

There is a paucity of information about the extent of polymorphism and evolution of the outer membrane protein genes including the SAG1 gene of S. neurona. Therefore, the objectives of this study were to examine the molecular basis of genetic variation and adaptive molecular evolution of the SAG1 gene of S. neurona and to identify amino acid sites of this gene under diversifying selection. The main findings of this study were the evidence that parts of the S. neurona SAG1 gene have evolved under positive selection and that the SAG1 gene has two alleles in the limited number of S. neurona isolates examined.

All SAG1 sequences used in the study were obtained from GenBank. Nine SAG1 sequences of S. neurona (Accession no. AF401682 (SEQ ID NO: 1), AF397896, AY032845, AY245695, AY170900, AF480854, AY170620 (SEQ ID NO: 2), AF480853, AY245696) were used as in-groups. One Toxoplasma gondii SAG1 sequence (Accession no. AY217784) was used as an out-group to root the phylogenetic tree(s). Nucleotide sequences were aligned using the multiple sequence alignment program Clustal X (Thompson et al. Nucleic Acids Res. 25, 4876-882, 1997) with the default parameters and checked by eye. Base frequencies and pairwise sequence divergences were obtained using the program PAUP* version 4.0b10 (Swofford, PAUP*, Phylogenetic Analysis Using Parsimony (* and Other Methods), Version 4. Sinauer Associates, Sunderland, Massachusetts, 2002). All insertion-deletion events were coded as missing data (“?”) for the purpose of phylogenetic analysis. Phylogenetic trees were sought with both maximum parsimony (MP) and neighbor-joining (NJ) using MEGA (Molecular Evolutionary Genetics Analysis, version 1.01) software (Kumar et al. Comput. Appl. Biosci. 10,189-91, 1994). NJ searches were performed to assess the influence of alternative phylogenetic algorithms on the tree topology. To find the shortest (MP) tree, a branch-and-bound search was performed with unknown initial upper bound and “furthest” taxa addition sequence. For the NJ search, the Kimura 2-parameter distance model was used. Relative bootstrap support for nodes in resulting trees (both by MP and NJ) was evaluated using 1000 pseudoreplicates (Felsenstein, Evolution 39, 783-791, 1985).

Aligned sequences used for phylogeny reconstruction consisted of 1685 bp in length. Of these 852 (51%) were variable and 208 (12%) were parsimony informative. Mean base proportions were 20.9% A, 22.9% T, 28.2% C, and 27.9% G. Chi-square test of homogeneity of base frequencies across taxa showed slight non-significant bias to the nucleotides G and C; 334.36 A, 366.65 T; 451.30 C, and 447.69 G; Chi-square=23.766 (df=24), P=D.4749. Uncorrected pairwise sequence divergence (p) across all taxa is given in Table 1.

TABLE 1
DNA sequence differences of SAG1 gene of Sarcocystis neurona isolates based
on pairwise comparisons using uncorrected (“p”) distance matrix.

| Isolate | Accession No | 1 | 2 | 3 | 4 | 5 | 6 | 7 | 8 | 9 |
|---|---|---|---|---|---|---|---|---|---|---|
| Sn-mucat2 | (AY245695) | — | | | | | | | | |
| Sn-mucat2 | (AY170900) | 0.000 | — | | | | | | | |
| UCD1 | (AF401682) | 0.000 | 0.000 | — | | | | | | |
| UCD1 | (AF397896) | 0.000 | 0.000 | 0.000 | — | | | | | |
| SN3 | (AY032845) | 0.000 | 0.000 | 0.000 | 0.000 | — | | | | |
| CAT2 | (AF480854) | 0.000 | 0.001 | 0.000 | 0.001 | 0.001 | — | | | |
| MU-1 | (AF480853) | 0.243 | 0.237 | 0.243 | 0.238 | 0.238 | 0.238 | — | | |
| Sn-MU1 | (AY245696) | 0.242 | 0.241 | 0.243 | 0.242 | 0.242 | 0.243 | 0.000 | — | |
| Sn-Mu1 | (AY170620) | 0.255 | 0.393 | 0.240 | 0.335 | 0.402 | 0.240 | 0.009 | 0.000 | — |

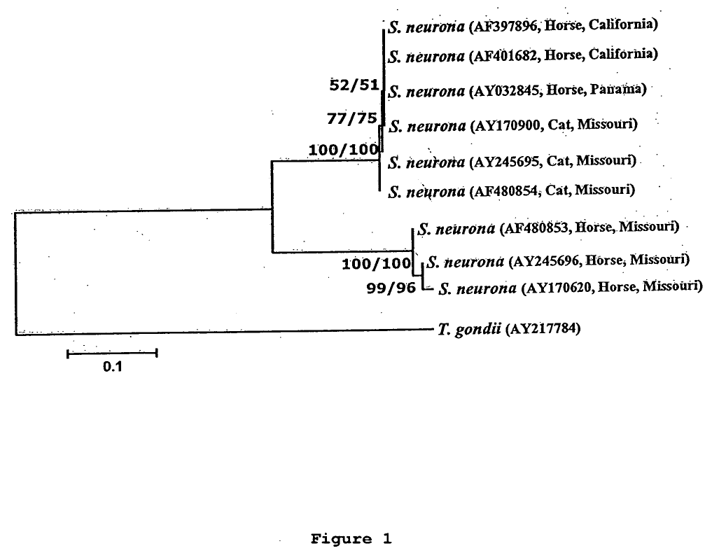

Sequences were clustered into two groups based on pairwise sequence divergence. The degree of divergence between the two groups was large but minimal within each group (Table 1). Nucleotide similarity analysis revealed that within each monophyletic group of S. neurona isolates, sequences of the SAG1 gene were 100% identical while sequence similarity between the two groups was 68.4%. A branch-and-bound search with equally weighted data produced a single tree, which was rooted by the T. gondii sequence. The SAG1 gene tree (FIG. 1) indicates clearly that there are two well-defined clusters. Cluster I consisted of six nucleotide sequences of three S. neurona isolates: SN3 and UCD1 from horses from Panama and California, respectively and CAT2 from cat from Missouri. Cluster II included three S. neurona sequences of isolate Sn-MUl from Missouri. Bootstrap values for NJ and MP methods obtained from 1000 replications were robust, with 100% support at the basal node of each lade. The fact that sequences within each cluster were identical and very different between the two clusters clearly indicates that there are two alleles of the SAG1 gene, which are referred to as H1 and H2, herein. The tree obtained by the NJ method had identical branching patterns and very close bootstrap support to the tree found by the MP method (FIG. 1). Kimura 2-parameter distance was used, although analyses with different types of distance models yielded the same tree. Maximum likelihood and Bayesian phylogenetic analyses of the nucleotide sequences also consistently yielded trees with almost identical topology, indicating that the inferred phylogenetic relationships are robust to different methods of tree estimation.

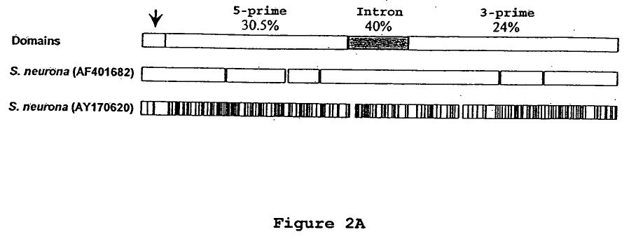

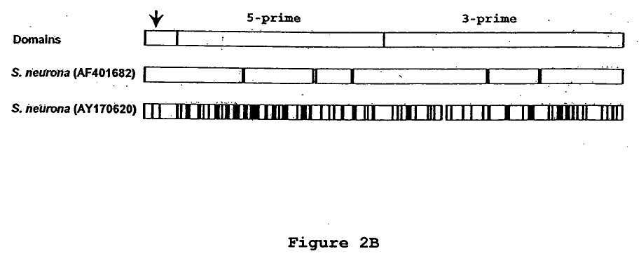

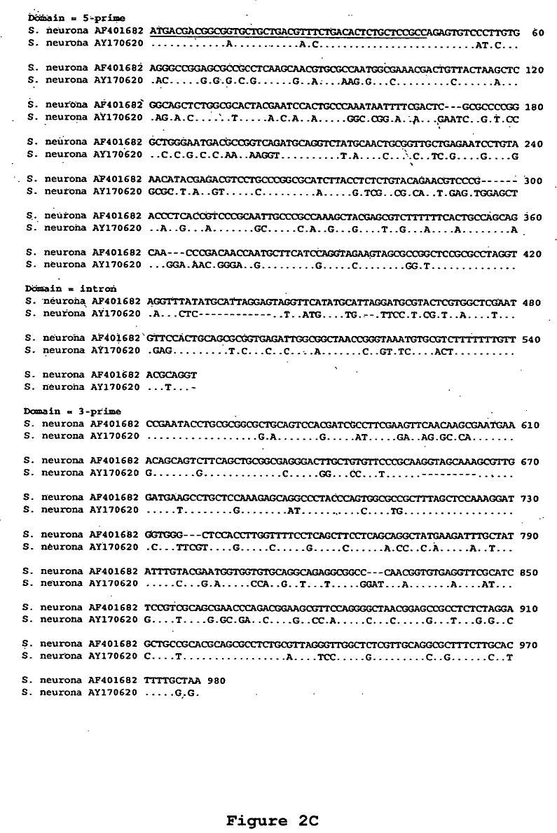

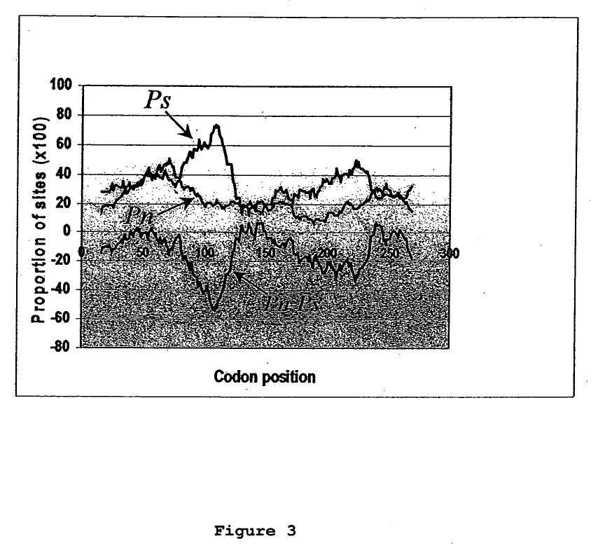

The numbers of polymorphic sites in the nucleotide and amino acid sequences of each allele were computed using the programs PSFIND (ver. 2) and PAFIND (ver. 1.1), respectively. These statistics were then used to draw the similarity plot (FIGS. 2A, 2B) using Happlot program (ver. 1.1). These three programs were written and kindly provided by Dr. Thomas S. Whittam (Microbial Evolution Laboratory, National Food Safety and Toxicology Center, Michigan State University). The degree of nucleotide and amino acid polymorphism and divergence between H1 and H2 alleles was remarkable. Variations in sequences were distributed evenly almost across the whole SAG1 gene (FIGS. 2A-2C). This gene showed much less constraint on amino acid-altering mutations among alleles. The ratio of nonsynonymous to synonymous mutations in the divergence of the whole gene between the H1 and H2 alleles was 0.73±0.05, a ratio that would be predicted for unconstrained, neutral variation. Two exceptions were noticed. First, there were two large segments where the per-site nonsynonymous-substitution rate peaked upward (FIG. 3). These peaked regions of the gene showed the most polymorphism as indicated in FIGS. 2A and 2B. Secondly, the region encoding the signal peptide (FIG. 3) showed the least polymorphism, where amino acid replacements were highly constrained in this part of the gene.

Additional measures of DNA sequence polymorphism and population genetic parameters such as the number of segregating sites, nucleotide diversity (π); average nucleotide differences (K), Theta (θ), number of haplotypes, haplotype diversity, and F-statistics were computed using the DnaSP program Version 3.52 (Rozas et al. Bioinformatics 15, 174-5, 1999). π is the average number of nucleotide differences per site between two sequences. Within each species, Tajimas's (Tajima, Genetics 123,585-95, 1989) test generated a beta-distributed parameter indicating the difference in two estimates (polymorphic sites and number of alleles) of diversity. Significantly low statistics indicated non-neutral evolution. Gaps and a few sites with unreliable information were coded as missing and ignored. The number of haplotypes was two and haplotype diversity (expected allele heterozygosity) was 1.0 and its variance (=0.25) and standard deviation (=0.5). The number of polymorphic (segregation) sites, s, was 204. Total number of mutations, Eta, was 23. The number of singleton segregating sites, Eta(s) was 35. This dataset had nucleotide diversity (per site), Pi (0.24818), its variance (=0.0000298), and standard deviation (=0.00546). The average number of pairwise nucleotide differences, K, was 204. Theta (per SAG1 gene) from Eta(s) was 204 and theta (per site) from Eta(s) was 0.368. Because selection may have played a role in the evolution of the SAG1 gene of S. neurona and divergence between isolates (given the assumption of a short divergence time), polymorphism data for each allele were tested by Tajima (Tajima, Genetics 123,585-95, 1989), and Fu and Li (Fu et al. Genetics 133,693-709, 1993) neutrality tests. Tajima's test compares two estimates of nucleotide diversity: the muataion parameter (θ) and the difference between pairs of isolates (n). Fu and Li's test is related to Tajima's and compares the number of singletons to that expected by the neutral theory and was performed without an out-group. Both tests rejected the neutral model of molecular evolution. Tajima's D was 0.539 (P>0.10). Fu and Li's D* and Fu and Li's F* test statistics were 0.493 and −0.26513 (P>0.10). Fu's Fs statistic was −2.971 (P>0.10).

Two hypotheses can be suggested to explain the high variability of the SAG1 gene of S. neurona based on these results. First, high levels of recombination are occurring between different lineages. Recombination could be a major source of variation that might have created the new SAG1 allele and generated putative antigenic diversity. To investigate this,. the degree of linkage disequilibrium (or nonrandom association between variants of different polymorphic sites) was estimated using the DnaSP program, with the following parameters: D, D′, R, and R2. Both the two-tailed Fisher's exact test and the chi-square test were computed to determine whether the associations between polymorphic sites are, or are not, significant. A moderate level of linkage disequilibrium was detected, which provide evidence for the possibility of frequent SAG1 recombination among these lineages. The second hypothesis, for. which there is no current supporting evidence, is that this gene might undergo “unconstrained evolution” through the rapid fixation of neutral mutations by genetic drift. In either case, the role of natural selection is problematic: in some parts of the molecule, it operates as a conservative force against amino acid change, whereas elsewhere it can be a diversifying force promoting protein polymorphism and rapid evolution.

The pattern of distribution of S. neurona seropositive horses has been attributed to the presence of the definitive host (opossum) in the same locality, where a high prevalence of seropositive horses was always associated with areas of opossum abundance (Rossano et al. Prev. Vet. Med. 57, 7-13, 2003). In this study, genetically identical isolates were widely distributed throughout the United States without any particular geographic segregation or any association with opossum distribution. Although, the correlation between the prevalence of EPM in horses and prevalence of opossums does occur, these findings indicate that the pattern of opossum distribution does not have any correlation with the SAG1 allele frequency of S. neurona isolates. Thus, the presence of S. neurona isolates in the Northern and Southern United States that have the identical SAG1 allele clearly indicates that there is no correlation between genetic variation and geographic distance and opossum distribution.

However, the role of intermediate hosts and other potential vectors particularly long-distance travelers such as migrating birds and cats accompanying people can not be ignored. These intermediate hosts might play an important role in the distribution of S. neurona parasites and promoting their diversity across the United States. S. neurona can be considered a generalist parasite because of its long list of host species. However, it is unknown whether or not the alternating life cycle of S. neurona involving the passage of the parasite through opossums and different intermediate hosts could have any impact on the genetic diversity of S. neurona. Potential selection pressures could stem from differences in host immune responses to infection or to differences in the physical host environment. Likewise, we cannot eliminate differences in microhabitat usage between different intermediate host species as a potential selective pressure. Compatible with this hypothesis, which is strongly supported by the presence of two alleles for the SAG1 gene, the higher inter-allelic polymorphism may be the consequence of a positive selection pressure that favored the fixation of non-synonymous nucleotide substitutions versus synonymous nucleotide substitutions in the coding region of the SAG1 gene.

The wide sequence variation of SAG1 found in the present study, coupled with the fact that it is serologically immunodominant and involved in the interaction of S. neurona with host cell receptors (Marsh et al. Vet. Parasitol. 95, 143-54, 2001; Marsh et al. Parasitol. Res. 88, 501-6, 2002; Ellison et al. Int. J. Parasitol. 32, 217-25, 2002; Hyun et al. Vet. Parasitol. 112, 11-20, 2003), suggests that the SAG1 protein may be involved in evading attack by the host's immune system. Positive selection is very important for the evolution of a new or improved protein function, as it increases the probability that an advantageous mutation becomes established in a population. During the SAG1 gene evolution, different codons might have undergone different ratios of nonsilent to silent base substitutions (d_{N}/dₛ). Previous studies have shown that genes on which positive selection may operate can be identified by comparing synonymous substitution rates (pₛ) and nonsynonymous substitution rates (p_{N}). To test this hypothesis, the proportions of p_{N} and pₛ polymorphic sites of the protein coding region of the SAG1 gene were calculated by the method of Nei and Gojobori (Nei et al. Mol. Biol. Evol. 3, 418-26, 1986). To examine how the level of selective constraint varies along different parts of the SAG1 gene, nucleotide site differences were tabulated in a sliding-window analysis of 30 codons for the length of the gene (FIG. 3) using the program PSWIN (Version 1.1) written by Dr. Thomas S. Whittam.

The difference, p_{N}−pₛ, is a measure of the degree of selective constraint: the more negative the value, the less the contribution of replacement substitutions and the greater the contribution of synonymous substitutions. The zero-difference line indicates selectively neutral variation, where the per-site rates of synonymous and nonsynonymous substitutions are equal. A positive difference, where amino acid replacements exceed the silent substitutions, suggests the action of diversifying (positive) selection. Of the whole open reading frame (ORF) of the SAG1 gene, only two stretches corresponding to the central and the most 5′ regions of the gene were identified where there was a positive difference between the nonsynonymous- and synonymous-substitution rates. The longest stretch with p_{N}/pₛ>1 was ˜51 codons in length and runs from position 114 to position 165. In this region, the rate of substitution per 100 sites (corrected for multiple hits) is d_{N}=192±16 and dₛ=165±21. The second region extends from codon 222 to codon 259. In this region, the rate of substitution was d_{N}=296±15 and dₛ=255±19. Thus, the selective pressures acting on the SAG1 gene are moderately positive in these two parts. Each of these two regions had a significant excess of nonsynonymous substitutions and thus is a likely target for diversifying selection, where natural selection has favored the substitution of amino acids. Because these short segments lie within the major antigenic surface protein of the SAG1 gene, the evolutionary benefit to new variants may be the enhanced ability to escape host immunity and colonize new hosts. These two regions might mediate the interactions of the parasite with host cells and codify the organism virulence traits. The reason for the presence of selective pressure at these particular parts of the gene remains unclear. However, it is possible that positive selection pressure does occur at a larger part of the gene but that conformational constraint on the SAG1 molecule act to reduce the value of d_{N}/dₛ below the critical threshold, but there is currently no theory that allows such an effect to be quantified. The inference of sites under diversifying selection in the SAG1 gene of S. neurona should prompt further lab-based investigation on the structure and function of the SAG1 protein to identify the selective agents.

In spite of the few number of sequences analyzed herein, this analysis provided statistically significant support for the existence of sites in the SAG1 gene of S. neurona under diversifying selection pressure and presented the SAG1 gene as an example of adaptive evolution. Positive selection analysis of genes coding for cell surface proteins of the malaria parasite, Plasmodium spp., have identified gene regions that have nonsynonymous substitution rates that are significantly higher than the synonymous rate (Ohta Genetics, 127, 345-53, 1991). Since host antibodies target these protein regions, it has been suggested that this phenomenon is evidence of positive selection-based diversification of the protein to maintain an advantage over the host immune system. Thus, given the fact that the SAG1 is serologically immunodominant, possibly this protein is important in allowing the parasite to evade the host immune response and the selective pressure is presumably the surveillance of the host immune system. In this event, the support for positive selection pressure on the SAG1 is consistent with the biological function of this gene and suggests that the SAG1 protein could be a good candidate antigen for vaccination against S. neurona infection. It is unclear if the genetic differences among S. neurona isolates are due to selection pressure exerted from the host species or from the off-host environment; both factors are likely to play a role. Controlled preference and cross-infection experiments, and further genetic comparisons between S. neurona populations of sympatrically occurring host species may provide some elements to help distinguish among these different possible forces. These findings have major implications for the design and interpretation of population genetic studies of selection on S. neurona genes.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

1. A diagnostic test kit comprising:
(a) one or more primary antibodies each specific for a subtype of a surface antigen gene 1 (SAG1) gene of Sarcocystis neurona; and
(b) one or more secondary antibodies conjugated to a particular label which are capable of binding the primary antibodies.

2. The diagnostic test kit of claim 1 wherein the label is selected from the group consisting of alkaline phosphatase, horseradish peroxidase, fluorescent compounds, luminescent compounds, colloidal gold, radioisotopes, biotin, colored latex, and magnetic particles.

3. The diagnostic test kit of claim 1 wherein the one or more primary antibodies are selected from the group consisting of monoclonal antibodies, polyclonal antibodies, and mixtures thereof.

4. The diagnostic test kit of claim 1 wherein the one or more primary antibodies are provided by a hybridoma.

5. The diagnostic test kit of claim 1 wherein the one or more antibodies are provided from serum.

6. A diagnostic test kit comprising:
(a) one or more polynucleotides specific for subtypes of the surface antigen gene 1 (SAG1) gene of Sarcocystis neurona; and
(b) hybridization solution for the binding of the one or more polynucleotides to a sample nucleic acid.

7. The diagnostic test kit of claim 6 wherein the one or more polynucleotides are selected from the group consisting of oligonucleotides, cDNAs, riboprobes, and mixtures thereof.

8. A PCR based diagnostic test kit comprising:
(a) one or more oligonucleotides specific for a coding region of the surface antigen gene 1 (SAG1) gene of Sarcocystis neurona;
(b) one or more oligonucleotides specific for a noncoding region of the surface antigen gene 1 (SAG1) gene of Sarcocystis neurona; and
(c) a thermostable DNA polymerase enzyme.

9. A method of diagnosing a sample from a horse with equine protozoal myeloencephalitis comprising:
(a) determining a nucleotide sequence of the surface antigen gene 1 (SAG1) gene of Sarcocystis neurona in the sample;
(b) comparing the nucleotide sequence with nucleotide sequences of known surface antigen gene 1 (SAG1) subtypes; and
(c) classifying the sample into a Sarcocystis neurona subtype.

10. The method of claim 9 wherein the nucleotide sequence is determined by DNA sequencing.

11. The method of claim 9 wherein the nucleotide sequence is determined by nucleic acid hybridization.

12. The method of claim 9 wherein the nucleotide sequence is determined by amplification by polymerase chain reaction (PCR) to produce a DNA followed by cutting of the DNA with a restriction enzyme which recognizes a sequence of interest.

13. A method of diagnosing a sample from a horse with equine protozoal myeloencephalitis comprising:
(a) determining an amino acid sequence of the surface antigen gene 1 (SAG1) gene of Sarcocystis neurona in the sample;
(b) comparing the amino acid sequence with amino acid sequences of known surface antigen gene 1 (SAG1) subtypes; and
(c) classifying the sample into a Sarcocystis neurona subtype to diagnose the protozoal myeloencephalitis.

14. The method of claim 13 wherein the amino acid sequence is determined by protein sequencing.

15. The method of claim 13 wherein the amino acid sequence is determined by antibody binding.

16. A vaccine for active immunization against Sarcocystis neurona comprising one or more polypeptides encoded by a location of positive selection within the surface antigen gene 1 (SAG1) gene of Sarcocystis neurona.

17. A vaccine for active immunization against Sarcocystis neurona comprising one or more polynucleotides having a sequence of a location of positive selection within the surface antigen gene 1 (SAG1) gene of Sarcocystis neurona.

18. A method of vaccine design comprising:
(a) determining a region of positive selection within the surface antigen gene 1 (SAG1) gene of Sarcocystis neurona; and
(b) selecting one or more polypeptides encoded by the region of positive selection within the surface antigen gene 1 (SAG1) gene to provide the vaccine.

19. A method of vaccine design comprising:
(a) determining a region of positive selection within the surface antigen gene 1 (SAG1) gene of Sarcocystis neurona; and
(b) selecting one or more polynucleotides having a sequence of a region of positive selection within the surface antigen gene 1 (SAG1) gene to provide the vaccine.

20. A method for making a prognosis related to Sarcocystis neurona infections in clinically infected horses comprising:
(a) determining a nucleotide or amino acid sequence of the surface antigen gene 1 (SAG1) gene of Sarcocystis neurona in the sample;
(b) comparing the nucleotide sequence with nucleotide sequences of known surface antigen gene 1 (SAG1) subtypes;
(c) classifying the sample into a Sarcocystis neurona subtype; and
(d) predicting the outcome of the infection according to the classification of the Sarcocystis neurona subtype.

21. A vaccine for immunization of a mammal against Sarcocystis neurona comprising a polynucleotide which encodes for a surface antigen gene 1 (SAG1) subtype of Sarcocystis neurona.

22. The vaccine of claim 21 wherein the polynucleotide is operably linked to a promoter which is functional in the mammal.

23. A method for eliciting an immune response in an equid against Sarcocystis neurona comprising:
(a) providing in a carrier solution a polynucleotide sequence encoding for a surface antigen gene 1 (SAG1) subtype of Sarcocystis neurona; and
(b) inoculating the equid with the polynucleotide sequence in the carrier solution to illicit the immune response.

24. The method of claim 23 wherein the polynucleotide is operably linked to a promoter which is functional in the mammal.

25. The method of claim 23 wherein the polynucleotide is provided in a vector DNA capable of infecting the equid.

26. A diagnostic test kit comprising:
(a) one or more antibodies each specific for a subtype of surface antigen gene 1 (SAG1) of Sarcocystis neurona; and
(b) a signal generating reagent for detecting each of the antibodies bound to the surface antigen.

27. A method which comprises diagnosing a Sarcocystis neurons infection in an equid which comprises testing with the test kits of claims 1 or 26 to determine the presence of the SAG1 gene.
